(19) United States
(12) Patent Application Publication
(10) Pub. No.: US 2010/0145437 A1
(43) Pub. Date: Jun. 10, 2010

(54) Stent Design Allowing Extended Release of Drug and/or Enhanced Adhesion of Polymer to OD Surface

(75) Inventors: Timothy S. Girton, (US); Jeffrey S. Lindquist, (US); David M. Knapp, (US); Matt Heidner, (US); Daniel Gregorich, (US); Umang Anand, (US); Peter Edelman, (US); Matthew J. Miller, (US); Raed Rizq, (US); Jan Weber, Maastricht (NL); Tracee Eidenschink, Wayzata, MN (US); John J. Chen, (US); Jaykeep Y. Kokate, (US); Aparna Bhave, (US); Kent D. Harrison, (US); Graig L. Kveen, (US); Benjamin Arcand, (US); Michael Kuchling, (US)

Correspondence Address: VIDAS, ARRETT & STEINKRAUS, P.A., SUITE 400, 6640 SHADY OAK ROAD, EDEN PRAIRIE, MN, 55344, US

(73) Assignee: Boston Scientific Scimed, Inc., Maple Grove, MN (US)

(21) Appl. No.: 12/706,881

(22) Filed: Feb. 17, 2010

Related U.S. Application Data
(63) Continuation of application No. 11/857,736, filed on Sep. 19, 2007.

(51) Int. Cl. A61F 2/82

(52) U.S. Cl. 623/1.42

(57) ABSTRACT

The invention is directed to mechanisms and methods that reduce the delamination of a therapeutic agent from a stent. The mechanisms include holes (channels, wells, and other hole configurations), protrusions, sintered metal cores, clamps/staples, pins, and stainless steel shields.

### CROSS-REFERENCE TO RELATED APPLICATIONS

This application is a continuation of U.S. application Ser. No. 11/857,736 filed Sep. 19, 2007, the contents of which are incorporated herein in its entirety by reference.

### STATEMENT REGARDING FEDERALLY SPONSORED RESEARCH

Not Applicable

### BACKGROUND OF THE INVENTION

1. Field of the Invention

In some embodiments this invention relates to implantable medical devices, their manufacture, and methods of use. Some embodiments are directed to delivery systems, such as catheter systems of all types, which are utilized in the delivery of such devices.

2. Description of the Related Art

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents, grafts, stent-grafts, vena cava filters, expandable frameworks, and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. They may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

Within the vasculature, it is not uncommon for stenoses to form at a vessel bifurcation. A bifurcation is an area of the vasculature or other portion of the body where a first (or parent) vessel is bifurcated into two or more branch vessels. Where a stenotic lesion or lesions form at such a bifurcation, the lesion(s) can affect only one of the vessels (i.e., either of the branch vessels or the parent vessel) two of the vessels, or all three vessels. Many prior art stents however are not wholly satisfactory for use where the site of desired application of the stent is juxtaposed or extends across a bifurcation in an artery or vein such, for example, as the bifurcation in the mammalian aortic artery into the common iliac arteries.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

### BRIEF SUMMARY OF THE INVENTION

In at least one embodiment, the invention is directed to mechanisms that reduce the delamination of a therapeutic agent from a stent. The mechanisms include various means to enhance mechanical interlocking between the stent surface and the therapeutic agent/coating. For example, in some embodiments the stent surface is provided with any of a variety of holes (channels, grooves, wells, and other hole configurations), protrusions and/or other surface features into and/or upon which various coatings or additional materials are engaged in a complementary fashion to provide an improved interlock between the coating (polymer or otherwise) and the stent surface. In one embodiment for example, the stent surface is provided with protrusions having a mushroom-like cross-sectional shape. A coating applied into the grooves between and around the protrusions will be engaged to the protrusions in the manner of a hook and loop material or VELCRO®. This is but one exemplary embodiment of the various interfaces possible between a coating and the stent surface in accordance with the present invention. These and several others are provided in greater detail below. Coating materials include but are not limited to, organic polymers, inorganic polymers, metal oxides, sintered metals, and/or others which may or may not also include an additional therapeutic agent or drug.

In at least one embodiment, the mechanisms engage different volumes of different therapeutic agents to the stent so that differential amounts of therapeutic agent are eluted from at least one member of the stent.

The invention is also directed to methods of engaging a therapeutic agent to a stent so that delamination of the therapeutic agent is reduced.

These and other embodiments which characterize the invention are pointed out with particularity in the claims annexed hereto and forming a part hereof. However, for further understanding of the invention, its advantages and objectives obtained by its use, reference can be made to the drawings which form a further part hereof and the accompanying descriptive matter, in which there is illustrated and described an embodiments of the invention.

### BRIEF DESCRIPTION OF THE SEVERAL VIEWS OF THE DRAWING(S)

A detailed description of the invention is hereafter described with specific reference being made to the drawings.

FIG. 1 is a perspective view of a stent comprising a plurality of members.

FIG. 2 is a top view of a portion of a member of the stent in FIG. 1.

FIG. 3 is a cross-sectional view of the member in FIG. 2 with two channels of equal depth.

FIG. 4 is a cross-sectional view of the member in FIG. 2 with three channels which have been overcoated with a therapeutic agent.

FIG. 5 is a cross-sectional view of the member in FIG. 2 with two channels of unequal depths.

FIG. 6 is a cross-sectional view of the member in FIG. 2 with channels on opposite sides of the member, where some of the channels have different widths.

FIG. 7 is a cross-sectional view of the member in FIG. 2 with channels on both sides of the member, thereby securing a therapeutic agent applied to the surface of the member between the channels.

FIG. 8 is a cross-sectional view of the member in FIG. 2 with surface bubbling that formed wells in the surface of the member.

FIG. 9 is a cross-sectional view of the member in FIG. 2 with oblique angle surface ablation resulting in holes that are at an oblique angle to the surface of the member.

FIG. 10 is a cross-sectional view of the member in FIG. 2 with a therapeutic agent held in place by V-shaped hole.

FIG. 11 is a perspective cross-sectional view of the member in FIG. 2 with a therapeutic agent held in place by a hole that extends through the body of the member from one surface of the member to the opposite surface of the member.

FIG. 12 is a perspective cross-sectional view of the member in FIG. 2 with cross-hatching.

FIG. 13 is a cross-sectional view of the member in FIG. 2 that delivers a therapeutic agent from a channel through an opening that has a smaller diameter than the channel.

FIG. 14 is a cross-sectional view of the member in FIG. 2 with a well protected by a lip.

FIG. 15 is a cross-sectional view of the member in FIG. 2 with a channel or well before a therapeutic agent is applied thereto.

FIG. 16 is a cross-sectional view of the member in FIG. 15 after the therapeutic agent has been applied and the edges of the channel have been pressed inwards to secure the therapeutic agent.

FIG. 17 is a top view of the member in FIG. 2 with a plurality of holes in the surface.

FIG. 18 is a cross-section of the member of FIG. 17 taken at line 18-18 showing a well or channel into which a therapeutic agent, that elutes through the holes, is deposited.

FIG. 19 is a cross-sectional view of the member in FIG. 2 with protrusions having a body and a cap region where the greatest width of the cap region is equal to the width of the body.

FIG. 20 is a cross-sectional view of the member in FIG. 2 with protrusions having a body and a cap region where at least a portion of the cap region has a width greater than the width of the body.

FIG. 21 is a cross-sectional view of the member in FIG. 2 with protrusions made of rough metal oxide engaged to the surface of the member.

FIG. 22 is a cross-sectional view of the member in FIG. 2 with edges extending from the surface of the member to form a channel for a therapeutic agent.

FIG. 23 is a cross-sectional view of the member in FIG. 2 with a therapeutic agent coating a stainless steel shield engaged to the member.

FIG. 24 is a top view of the stainless steel shield of FIG. 23.

FIG. 25 is a perspective cross-sectional view of the member in FIG. 2 with a porous core and holes extending therefrom.

FIG. 26 is a perspective cross-sectional view of a wire with a porous core and holes extending therefrom.

FIG. 27 is a perspective cross-sectional view of the member in FIG. 2 with a porous core and holes extending therefrom to only one side of the member.

FIG. 28 is a perspective cross-sectional view of the member in FIG. 2 with a porous coating around the body of the member, where the porous coating is surrounded by a polymer coating with holes therethrough.

FIG. 29 is a perspective cross-sectional view of the member in FIG. 2 with a porous core and holes with a larger diameter at the surface than at the core.

FIG. 30 is a perspective view of a portion of the member in FIG. 2 with a therapeutic agent deposited thereon and held in place on the member by a metallic clamp.

FIG. 31 is a perspective view of a portion of the member in FIG. 2 with staples engaged thereto that have a therapeutic agent deposited thereon.

FIG. 32 is a perspective view of the member in FIG. 2 with a pin inserted therein that has a therapeutic agent deposited thereon.

FIG. 33 is a cross-section of the member in FIG. 2 with a therapeutic agent deposited onto a passivation layer.

FIG. 34 is a cross-section of the member in FIG. 2 with a textured surface made by removing portions of the member surface.

FIG. 35 is the cross-section of FIG. 34 with core-shell particles deposited within the indentations of the textured stent surface.

FIG. 36 is a cross-section of the member in FIG. 2 with surface structures added to the surface of the member to form a textured surface.

FIG. 37 is the cross-section of FIG. 36 with core-shell particles deposited within the indentations of the coating forming the textured surface.

FIG. 38 is a cross-section of the member in FIG. 2 with a coating, where a portion of the coating has been removed to form a textured surface.

FIG. 39 is the cross-section of FIG. 38 with core-shell particles deposited within the indentations of the coating forming the textured surface.

FIG. 40 is a view of a mask to be used in a cylindrical lithography procedure.

FIG. 41 is an end view of the mask of FIG. 40 and a stent tube undergoing a cylindrical lithography process.

FIG. 42 is a side view of a stent tube of FIG. 41 where the pattern from the mask transferred onto the surface of the stent tube.

FIG. 43 is a cross-section of the stent tube of FIG. 42 prior to electropolishing.

FIG. 44 is the cross-section of the stent tube of FIG. 42 after electropolishing.

FIG. 45A-D are cross-sections of embodiments of core-shell particles containing at least one therapeutic agent.

FIG. 46 is a side view of the member in FIG. 2 with protrusions.

FIG. 47 is a side view of the member in FIG. 46 with a therapeutic agent applied thereto.

FIG. 48 is a side view of the member in FIG. 47 with the therapeutic agent covering the entire outer surface of the member, including the protrusions.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

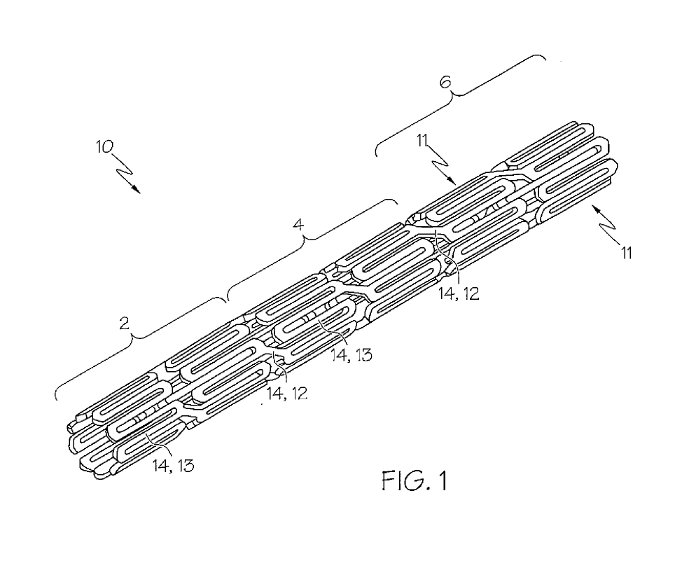

FIG. 1 depicts a stent 10 comprising a plurality of members 14 that form circumferential rings 11 that extend about the circumference of the stent 10. The stent 10 illustrated in FIG. 1 is an example of a configuration for a non-bifurcated stent 10. The stent 10 configuration in FIG. 1 is presented only as an example of one stent 10 configuration that can be used to deliver therapeutic regimens, any stent 10 configuration can be used including configurations for bifurcated stents 10. Stents 10 have different regions and/or subregions. As a non-limiting example, the stent 10 in FIG. 1 can be divided into a proximal region 2, a middle region 4 and a distal region 6, where each region has two circumferential rings 11 of members 14. One of ordinary skill in the art will recognize that there are numerous ways in which the stent 10 of FIG. 1 can be designed to have different regions and/or subregions that have different sizes and positions along the longitudinal length of the stent 10. Different regions and/or subregions of a bifurcated stent used to deliver different therapeutic regimens are discussed in greater detail in Bifurcated Stunt with Drug Wells for Specific Ostial, Carina, and Side Branch Treatment, Attorney Docket Number S63.2B-13099-US01, with inventors Dan Gregorich, Mike Meyer and Dave Friesen, hereby incorporated by reference herein in its entirety.

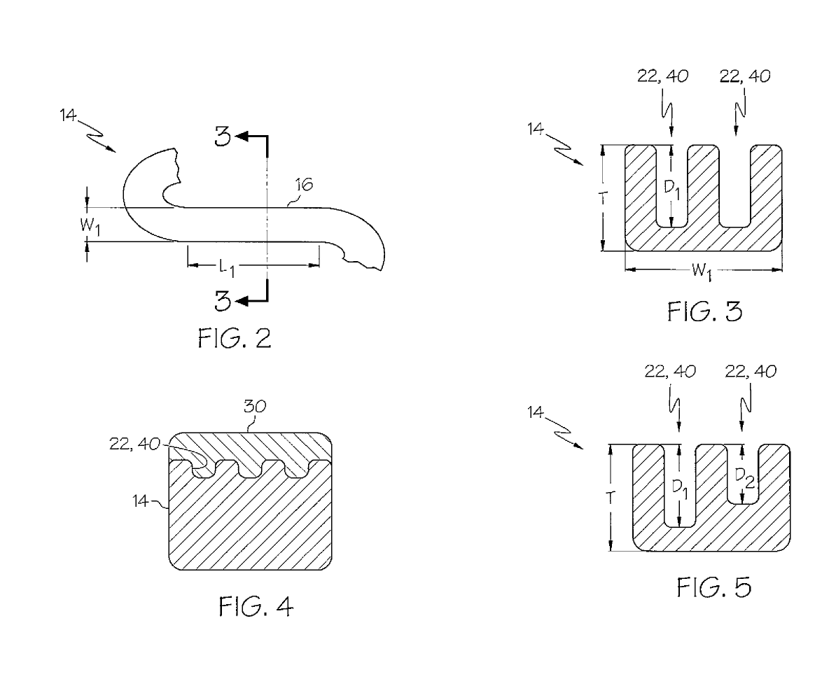

Members 14, as used in this application, include both struts 13 and connectors 12. Some of the members 14 have at least one straight section 16 and at least one turn 18. The straight section 16 of the member 14 may be the same width as the at least one turn 18 or may be wider than the at least one turn 18. Each member 14 has four sides from which therapeutic agents 30 can be eluted: the abluminal side (side of member 14 adjacent to the lumen wall), the luminal side (side of member 14 adjacent to the lumen) and the other two sides of the member 14 which are at an oblique angle to the luminal and abluminal sides of the member 14. As used in this application, an oblique angle is any angle between 0 and 180 degrees and includes 90 degrees. Each member 14 has a length (L₁), width (W₁) and depth (T), as shown in FIGS. 2 and 3.

In at least one embodiment, the stent 10 has one coating retainer 22. A coating retainer 22, as used in this application, is any means that reduces delamination of a substance, e.g. a therapeutic agent 30, from at least a portion of the stent 10. One means to reduce delamination is to increase the adhesion of the therapeutic agent 30 to the stent 10. Coating retainers 22 that increase the adhesion of the therapeutic agent 30 to the stent 10 include holes 44 (channels 40, wells 42 and V-shaped holes 46), protrusions with various cross-sectional shapes 48a-f, clamps 54/staples 56, pins 58, porous material 34, and any combination thereof. These different coating retainer 22 embodiments are discussed in greater detail below.

Note that some coating retainers 22 are engaged to the surface of the member 14, as shown, for example, in FIG. 19; some coating retainers 22 are positioned within the body of the member 14, as shown, for example, in FIG. 3; and some coating retainers 22 have a portion that is within the body of the member 14 and a portion extending from the surface of the member 14, as shown, for example, in FIG. 32. Coating retainers 22 engaged to the surface of the member 14 have at least one therapeutic agent 30 deposited onto the coating retainer 22 while coating retainers 22 within the body of the member 14 have at least one therapeutic agent 30 deposited within/into the coating retainer 22. Coating retainers 22 that have a portion within the body of the member 14 and a portion above the surface of the member 14 have at least one therapeutic agent 30 deposited on the portion of the coating retainer 22 that is above the surface of the member 14. Different methods of depositing the therapeutic agent 30 into/onto the coating retainers 22 are discussed in detail below.

It is within the scope of the invention for at least one of the members 14 of the stent 10 to have one, two, three, four, five, six, seven, eight, nine, ten, eleven, twelve, thirteen, fourteen, fifteen, sixteen, seventeen, eighteen, nineteen, twenty, or more coating retainers 22. In one embodiment, the stent 10 has only one type of coating retainer 22. It is within the scope of the invention for a stent 10 to have at least two different types of coating retainers 22. Thus one member 14 may have channels 40 while another member 14 has wells 42, while another member 14 has protrusions 48, etc. In one embodiment, different regions of the stent 10 have different types of coating retainers 22. In at least one embodiment, a member 14 of the stent 10 has at least two different types of coating retainers 22.

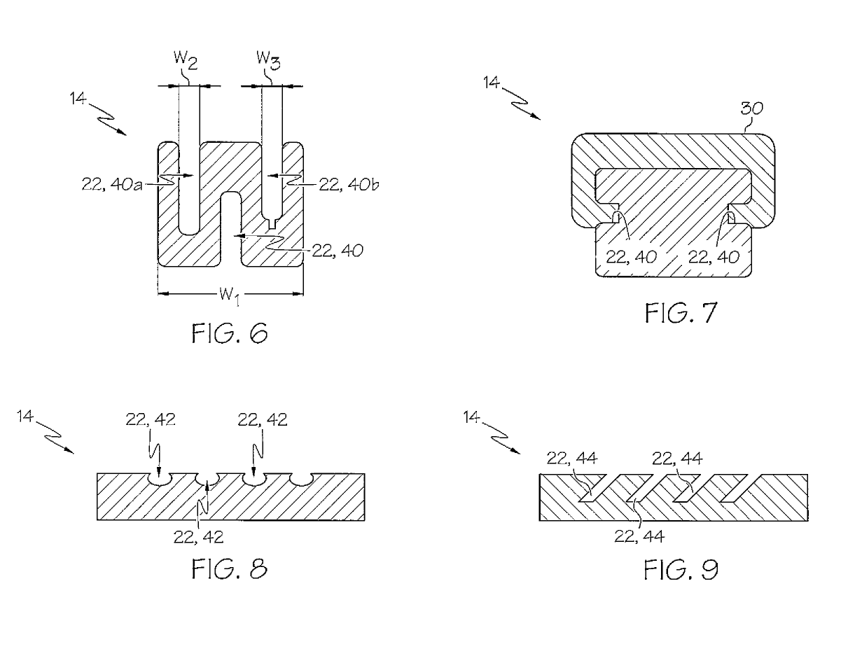

It is within the scope of the invention for at least one member 14 of the stent 10 to have coating retainers 22 on at least one side of the member 14. In at least one embodiment, the coating retainers 22 are located on the abluminal side of the members 14 of at least one region of the stent 10. In at least one embodiment, the coating retainers 22 are located on the luminal side of the members 14 of at least one region of the stent 10. In at least one embodiment, the coating retainers 22 are located on both the abluminal side and the luminal side of the member 14 of at least one region of the stent 10, as illustrated, for example, in FIG. 6. In at least one embodiment, the coating retainers 22 are located on only one side of the members 14 of at least one region of the stent 10. In at least one embodiment, the coating retainers 22 are located on all sides of the members 14 of at least one region of the stent 10. In at least one embodiment, the coating retainers) 22 on one side of the member 14 are different than the coating retainer(s) 22 on another side of the member 14.

Each of the coating retainers 22 have a length (L₂) width (W₂) and depth (D₁) and therefore a size or volume, which is affected by changes in at least one of these dimensions. However, the size/volume of the portion of the coating retainer 22 within the body of the member 14 should not be large enough to affect the integrity of the member 14. In addition the number and position of multiple coating retainers 22 with at least a portion of the coating retainer 22 positioned within the body of the member 14 should be configured so as not to affect the integrity of the member 14.

The depth (D₁) of a coating retainer 22 within the body of the member 14 is the distance from the opening of the coating retainer 22 to the bottom surface of the coating retainer 22, as illustrated for example in FIG. 3. Note that a coating retainer 22 can have at least two different depths, shown for example, in FIG. 5. In at least one embodiment, the coating retainers 22 have a length (L₂) which is at most equal to the length (L₁) of the straight section 16 of the member 14. In at least one embodiment, the coating retainers 22 have a length (L₂) which is at a minimum equal to ¼(L₁). Thus it is within the scope of the invention for the coating retainer 22 to have a length (L₂) ranging from ¼(L₁) to L₁. In at least one embodiment, the opposite sides of the coating retainer 22 have different lengths (L₂). This can occur, for example, if the ends of the coating retainer 22 are non-perpendicular to the sides of the coating retainer 22. Thus, the coating retainer has at least two different lengths. In at least one embodiment, the opposite sides of the coating retainer 22 have the same length (L₂). This occurs, for example, if the ends of the coating retainer 22 are perpendicular to the sides of the coating retainer 22. In at least one embodiment, the coating retainer 22 has a length (L₂) equal to ¼(L₁). In at least one embodiment the coating retainer 22 has a length (L₂) is equal to ½(L₁).

The width (W₂) of the coating retainer 22 is the distance from the opposite sides of the coating retainer 22. In at least one embodiment, the coating retainers 22 on a member 14 have the same width. In at least one embodiment, the coating retainers 22 on a member 14 have different widths. Thus, coating retainers 22 on the same side of a member 14 can have different widths, shown, for example, in FIG. 6 where W₂ is different from W₃. Similarly, coating retainers 22 on the same side of a member 14 can have a first width and the coating retainers 22 on another side of the member 14 can have a second width, where the first and second widths are different. In at least one embodiment, the coating retainer 22 has a variable width. The width can vary along the length of the coating retainer 22 or along the depth of the coating retainer 22, such as is seen for example, in FIG. 13.

In contrast to coating retainers 22 positioned within the body of the member 14, coating retainers 22 positioned on the surface of the member 14, can be engaged to the entire surface of the member 14 or to only a portion of the surface of the member 14 because coating retainers 22 positioned on the surface of the member 14 do not affect the integrity of the member 14. Thus, these “surface” coating retainers 22 can have the same configuration as the member 14, e.g. if the member 14 is curvilinear, the coating retainer 22 can be curvilinear, or these “surface” coating retainers 22 can have a different configuration than the member 14, e.g. if the member 14 is rectangular shaped, the coating retainer 22 can be round shaped. The depth (Dᵢ) of a coating retainer 22 engaged to the surface of the member 14 is measured from the surface of the member 14 to the top surface or highest point of the coating retainer 22, the highest point having the greatest distance from the surface of the member 14. In at least one embodiment, the coating retainer 22 engaged to the surface of the member 14 has a plurality of depths, or variable depths, shown for example in FIG. 19.

In at least one embodiment, the coating retainer 22 has at least one therapeutic agent 30 deposited therein/thereon. Multiple therapeutic agents 30 may be deposited as layers. For example, a first therapeutic agent 30 is deposited into/onto the coating retainer 22, then a second therapeutic agent 30 is deposited into/onto the coating retainer 22, thereby forming two layers of therapeutic agent 30. Each layer may contain the same volume of therapeutic agent 30 or different volumes of therapeutic agent 30. Layers may contain the same therapeutic agent 30 but the concentration of the therapeutic agent 30 in adjacent layers is different. The number of layers in/on the coating retainers 22 depends upon the depth of the coating retainer 22 and the depth of each layer of therapeutic agent 30. Thus, a coating retainer 22 can have any number of layers of therapeutic agent 30 desired.

Individual members 14 can elute multiple therapeutic agents 30 and/or different volumes of therapeutic agent 30. In at least one embodiment, one side of the member 14 has at least two different therapeutic agents 30 deposited in/on the coating retainers 22. In some embodiments, at least one of the coating retainers 22 on a member 14 has a different therapeutic agent 30 than the other coating retainers 22 on the member 14. In at least one embodiment, coating retainers 22 on different sides of the member 14 elute different therapeutic agents 30.

The volume of therapeutic agent 30 deposited in/on the coating retainer 22 corresponds to the length of time the therapeutic agent 30 elutes from the stent 10 so that a larger volume of therapeutic agent 30 elutes for a greater amount of time than a smaller volume of therapeutic agent 30. Therefore, the volume of therapeutic agent 30 deposited into/onto the coating retainers 22 can be optimized so that the therapeutic agent 30 elutes from the stent 10 for the desired amount of time. The layering and volumes of therapeutic agent 30 allows for the elution of the therapeutic agents 30 from the stent 10 in a desired sequence and for a desired amount of time. Note that other variables, for example the shape of the coating retainer 22, can also affect the elution rate of the therapeutic agent 30. The relationship between the elution rate and the shape of the cavity holding a therapeutic agent is discussed in greater detail in U.S. Pat. No. 6,709,379 to Brandau et al., which is hereby incorporated by reference in its entirety.

In at least one embodiment, the member 14 has a first coating retainer 22a and a second coating retainer 22b, and the first coating retainer 22a has a first volume of therapeutic agent 30 and the second coating retainer 22b has a second volume of therapeutic agent 30, where the first volume is different from the second volume. In at least one embodiment, the coating retainer 22 is overcoated with a therapeutic agent 30. In at least one embodiment, overcoating occurs when the coating retainer 22 is over filled so that the therapeutic agent 30 is deposited on the surface of the member 14 as well as in/on the coating retainer 22, as illustrated, for example, in FIG. 4. In at least one embodiment, overcoating occurs when the depth of the therapeutic agent 30 is greater than the height of the coating retainer 22, illustrated, for example, in FIG. 20.

The rate of elution is also affected by the use of coating barriers, which also modulate the elution, interactivity and/or effectiveness of a therapeutic agent 30 from the coating retainer 22. In at least one embodiment, the stent 10 has a coating barrier in/on the coating retainer 22. Coating barriers can be selectively used on at least one region of the stent 10 depending upon the desired therapeutic regimen. Thus, for example, the proximal region 2 of the stent 10 can have a coating barrier while the distal region 6 of the stent 10 does not have a coating barrier. The combinations of coating barrier and stent region are numerous and are contemplated as being within the scope of the invention. In addition, coating barriers can be used within an individual coating retainer 22 so that a coating barrier separates at least two volumes of therapeutic agent 30 in/on the coating retainer 22.

The coating barriers can be permeable, variably permeable, or impermeable. The permeability of the variably permeable barriers can vary over time and can be due to the degradation of the coating barrier due to a response to pH, salinity, temperature, current, or any other environmental factor. Examples of suitable coating barriers, include, but are not limited to, bioabsorbable materials, biodegradable materials and biostable polymers.

As used in this application, bioabsorbable also means biodegradable, degradable, biologically degradable, erodable, bioresorbable, and the like. The material used to inhibit the elution/diffusion of the therapeutic agent 30 dissolves, dissociates, or otherwise breaks down in the body without ill effect.

Examples of suitable bioabsorbable materials include, but are not limited to, poly(hydroxyvalerate), poly(L-lactic acid), polycaprolactone, poly(lactide-co-glycolide), poly(hydroxybutyrate), poly(hydroxybutyrate-co-valerate), polydioxanone, polyorthoesters, polyanhydrides, poly(glycolic acid), poly(D,L-lactic acid), poly(glycolic acid-co-trimethylene carbonate), polyphosphoesters, polyphosphoester urethanes, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen, hyaluronic acid, etc., and mixtures thereof.

As used herein, the term “polylactide” is equivalent to “poly (lactic acid)” as meaning a polymer of lactic acid. In particular, DL-lactide is a lactide derived from a roughly racemic mixture of lactic acid, and this nomenclature is interchangeable with (DL) lactic acid. Similarly, the terms polyglycolide and poly (glycolic acid) are equivalent.

Examples of biostable polymers include, but are not limited to, polystyrene-b-polyisobutylene-b-polystyrene block copolymer (SIBS), Poly-Butyl-Methacrylate (PBMA), and Polyvinylidene Difluoride (PVDF).

Other suitable materials that can be used as coating barriers can be found in U.S. Pat. No. 5,358,475, entitled High Molecular Weight Bioresorbable Polymers and Implantable Devices Thereof, U.S. Pat. No. 7,070,616, entitled Implantable Valvular Prosthesis, and U.S. Patent Application Publication No. 2005/0043816, entitled Reticulated Elastomeric Matrices, Their Manufacture and Use in Implantable Devices, each of which are incorporated herein in their entirety.

As mentioned above, there are many different types of coating retainers 22 that can decrease delamination of a therapeutic agent 30 from the stent 10. Different types of coating retainers 22 are illustrated in FIGS. 3-32. Many of the figures are a cross-sectional view of the member 14 in FIG. 2 taken at line 3-3, across the width (W₁) of member 14. FIG. 2 is a top view of a member 14 of the stent 10 in FIG. 1 with a straight section 16.

Coating retainers 22 positioned within the body of the member 14 are illustrated in FIGS. 3-18. One type of coating retainer 22 positioned within the body of the member 14 are holes 44. Holes 44 can be blind holes in the shape of channels 40, as shown in FIGS. 3-7, wells 42, as shown in FIG. 8, or other configurations, as shown for example in FIGS. 9-10 and 12-18. A blind hole 44, as used in this application, is a hole 44 extends partway through the body of the member 14, so that the depth of the hole 44 is less than the thickness of the member 14, as shown, for example, in FIG. 9. Alternatively holes 44 can be through-holes 44, as shown, for example, in FIG. 11. Unlike a blind hole 44, a through-hole 44 extends from one surface of the member 14, through the body of the member 14 to another surface of the member 14, typically the opposite surface. To differentiate between the different types of holes 44, reference will be made to the specific types of holes 44, e.g. channel 40, well 42, hole 44, V-shaped holes 46, and through-hole 44.

In at least one embodiment, the holes 44 are in the shape of channels 40. In at least one embodiment, therapeutic agent 30 deposited in the channels 40 enhance the adhesion of therapeutic agent 30 on the surface of the member 14. In at least one embodiment, the therapeutic agent 30 deposited within the channel 40 is different than the therapeutic agent 30 deposited on the surface of the member 14.

Each channel 40 has four sides, a bottom surface and an opening and extends along the length (L) of at least one side of the straight section 16 of the member 14. In at least one embodiment, the coating retainers 22 on a member 14 have the same depth, as illustrated, for example, in FIG. 3. In at least one embodiment, the coating retainers 22 on a member 14 have different depths, and therefore different volumes. In at least one embodiment, the coating retainers 22 on a side of the member 14 have two different depths (D₁ and D₂), as illustrated, for example, in FIG. 5.

In at least one embodiment, a channel 40b has two different depths, as illustrated, for example, in FIG. 6 or FIG. 12. In FIG. 6 there is a channel in the bottom surface of the channel 40b. In one embodiment, the sides of the channel in the bottom surface of the channel 40b follows the sides of the channel 40b, for example where the channel 40b has straight longitudinal sides and the channel in the bottom surface also has straight longitudinal sides. In one embodiment, the channel in the bottom surface of the channel 40b is a zig-zag channel. In at least one embodiment, therapeutic agent 30 is deposited within the first channel 40 and the second channel 40 in the bottom surface of the first channel 40, so that the therapeutic agent 30 deposited within the second channel 40 reduces delamination of the therapeutic agent 30 deposited within the first channel 40.

The width of a channel 40 is the distance from the opposite sides of the channel 40. Note that the sides determining the width of the channel 40 are at an oblique angle to the sides of the channel 40 that are used to determine the length of the channel 40. In at least one embodiment, the sides determining the width of the channel 40 are at right angles, i.e. 90°, to the sides determining the length of the channel 40. In at least one embodiment, the coating retainers 22 on a member 14 have the same width. In at least one embodiment, at least one coating retainer 22 has a different width (W₂), as illustrated in FIG. 6 where W₂ is different from W₃. In at least one embodiment, the coating retainers 22 on the same side of a member 14 have a first width and the coating retainers 22 on another side of the member 14 have a second width, where the first and second widths are different. In at least one embodiment, the width of the opening of the channel 40 is less than the width of the bottom surface of the channel 40. Thus in this embodiment, the channel 40 has at least two widths. The smaller width of the opening reduces delamination of the therapeutic agent 30 deposited within the channel 40.

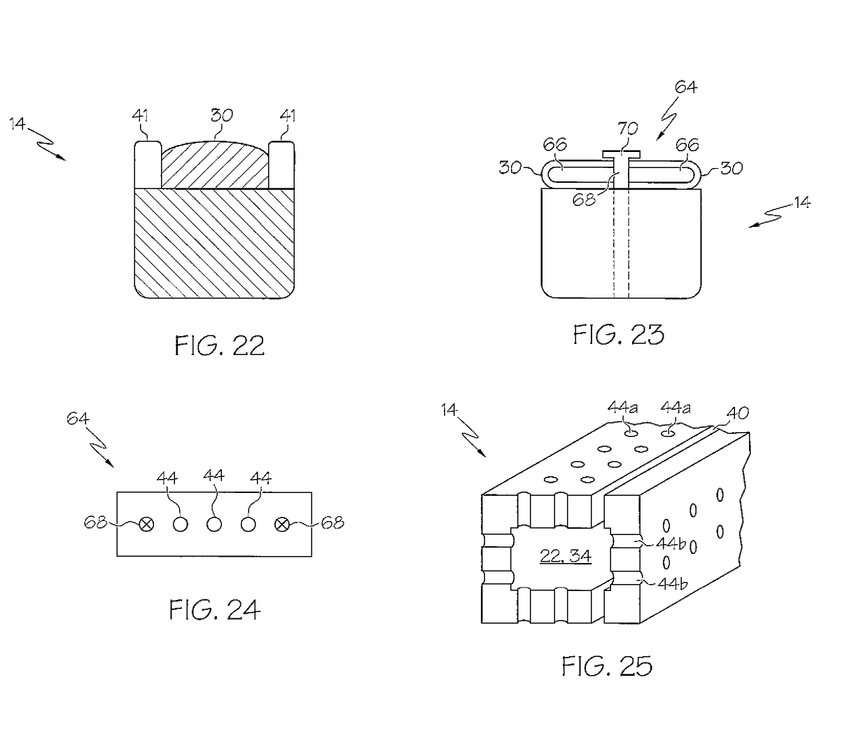

In FIG. 7, the channels 40 are positioned on opposite sides of the member 14. In this embodiment, therapeutic agent 30 deposited within the channels 40 in the member 14 enhances the adhesion of the therapeutic agent 30 engaged to the surfaces of the member 14 between the two channels 40. Note that the channels 40 do not have to extend into the body of the member 14, as illustrated in FIGS. 3-7 but can be on the surface of the member 14, as illustrated for example in FIG. 22 and discussed in greater detail below.

In at least one embodiment, the holes 44 are in the shape of wells 42, shown, for example, in FIG. 8. As discussed above, wells 42 are one type of blind hole 44. In at least one embodiment, therapeutic agent 30 deposited within the wells 42 increases the adhesion of the therapeutic agent 30 on the surface of the member 14. In at least one embodiment, the therapeutic agent 30 deposited within the wells 42 is different than the therapeutic agent 30 deposited on the surface of the member 14. It is within the scope of the invention for the wells 42 to have any size and configuration and to be positioned along at least one portion of the surface of the member 14 or along the entire surface of the member 14. Methods to manufacture the wells 42 into the surface of the member 14 include grit blasting, and ion beam etching, which are described in greater detail below. Note that with this embodiment, a cross-section of the length of the member 14 would look the same as the cross-section of the width of the member 14, shown in FIG. 8.

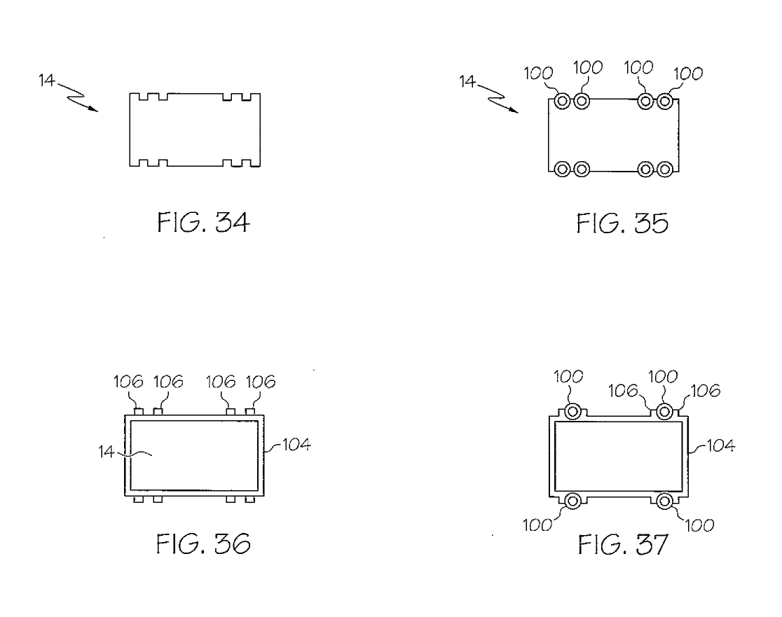

In addition to forming channels 40 and wells 42 in the surface of the member 14, irregular features/indentations can be formed in the surface of the member 14 to decrease delamination of the therapeutic agent 30 deposited onto the surface of the member 14. Methods of forming these irregular features/indentations are discussed in greater detail below. Therapeutic agent 30 deposited into/onto the irregular features/depressions made in the surface of the member 14 enhances the adhesion of therapeutic agent 30 deposited on the surface of the member 14. In at least one embodiment, core-shell therapeutic agent 30 containers are deposited into the depressions, as discussed in greater detail below in reference to FIGS. 34-39.

Other embodiments of coating retainers 22 with holes 44 are illustrated in FIGS. 9-12. In these embodiments, therapeutic agent 30 deposited within the holes 44 increases the adhesion of therapeutic agent 30 on the surface of the member 14. In at least one embodiment, the therapeutic agent 30 deposited within the holes is different than the therapeutic agent 30 deposited on the surface of the member 14.

Note that although embodiments with holes 44 are shown having substantially round shaped openings, the openings can have any configuration, for example, but not limited to, square shaped, rectangular shaped, oval shaped, oblong shaped, bow-tie shaped, X-shaped, polygonal shaped, irregular shaped, and any combination thereof. The passageway of the holes 44 can have the same configuration as the opening, a different configuration from the opening or more than one configuration.

FIG. 9 is an example of coating retainers 22 which are blind holes 44 that extend into the body of the member 14. In this embodiment, the holes 44 are at an oblique angle to the surface of the member 14. As discussed above, an oblique angle is an angle between 0 and 180 degrees and includes 90 degrees. Note that with this embodiment, a cross-section of the length of the member 14 would look the same as the cross-section of the width of the member 14, shown in FIG. 9. In at least one embodiment, therapeutic agent 30 is deposited into the holes 44 and onto the surface of the member 14. In this embodiment, the therapeutic agent 30 deposited into the holes 44 reduces delamination of the therapeutic agent 30 deposited onto the surface of the member 14. Different methods to make the holes 44 are described in greater detail below.

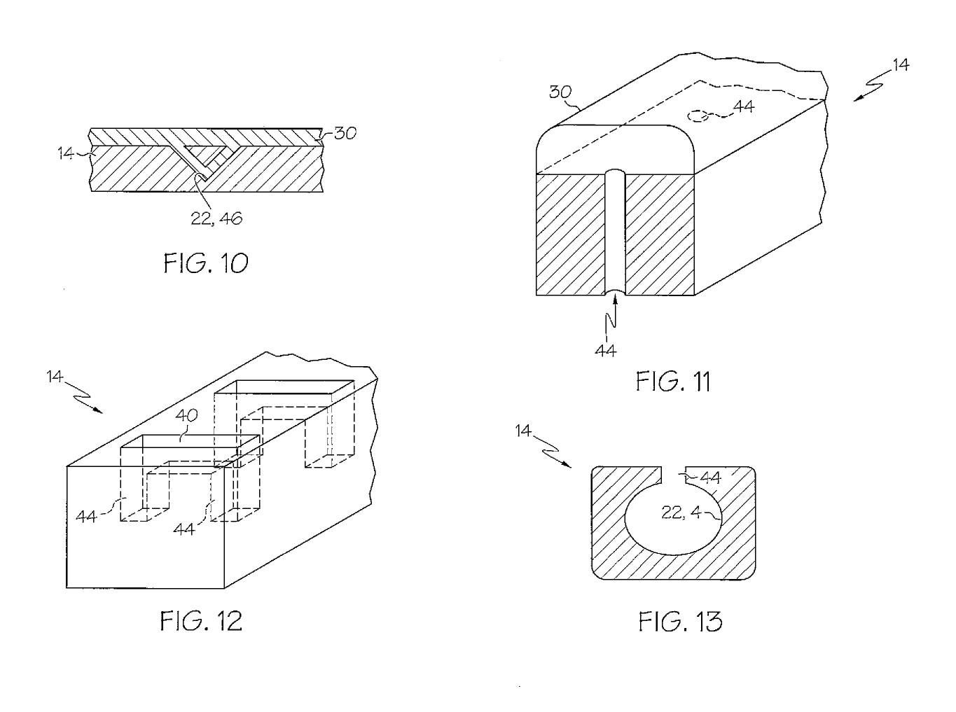

Another type of coating retainer 22 is formed by at least two holes 44 that extend into the body of the member 14 at oblique angles to at least one surface of the member 14 and intersect one another. One example is a V-shaped hole 46, shown in FIG. 10. It is within the scope of the invention for the passageway formed by the intersection of the at least two holes 44 to have any shape, for example, but not limited to, V-shaped, U-shaped, Y-shaped, y-shaped, X-shaped, L-shaped, T-shaped, irregular shaped and any combination thereof. For simplicity, the term “V-shaped hole 46” is used in this application to refer any configuration of this type of coating retainer 22. As shown in FIG. 10, the V-shaped hole 46 has a V-shaped passageway. Although, the two holes 44 forming the V-shaped hole 46 in FIG. 10 both have openings on the same surface of the member 14, it is within the scope of the invention for the two holes 44 forming the V-shaped hole 46 to have openings on different surfaces of the member 14. This type of V-shaped hole 46 can have an L-shaped passageway.

In at least one embodiment, at least one of the holes 44 is a through hole. Examples of configurations where at least one of the holes 44 is a through hole include, but are not limited to V-shaped, y-shaped, X-shaped and T-shaped holes 46. A T-shaped hole 46 can have a through hole 44 extending between opposite sites of a member 14 and a hole 44, extending from a third side, intersecting the through hole 44 to form a T shaped passageway 46.

In at least one embodiment, the therapeutic agent 30 is deposited only within the V-shaped holes 46. In at least one embodiment, the therapeutic agent 30 is deposited within the V-shaped holes 46 and on the surface of the member 14. In this embodiment, the therapeutic agent 30 deposited within the V-shaped holes 46 helps prevent the delamination of the therapeutic agent 30 deposited on the surface of the member 14. Note that with this embodiment, a cross-section of the length of the member 14 would look the same as the cross-section of the width of the member 14, shown in FIG. 10. Thus, the V-shaped hole 46 can be parallel to the length of the member 14 or parallel to the width of the member 14 In at least one embodiment, the therapeutic agent 30 deposited into the V-shaped holes 46 is different than the therapeutic agent 30 deposited on the surface of the member 14. In at least one embodiment, each passageway of the V-shaped hole 46 has different therapeutic agents 30 deposited therein.

FIG. 11 is an example of a coating retainer 22 that is a through-hole 44 that extends from one surface of the member 14, through the body of the member 14, to the other surface of the member 14. The through-hole 44 has a first opening, a second opening and a passageway extending therebetween. In at least one embodiment, the through-hole 44 is perpendicular to the surfaces of the member 14. In at least one embodiment, the through-hole 44 is at an oblique angle to the surfaces of the member 14. In this embodiment, the through-hole 44 extends between opposite sides/surfaces of the member 14. Note that a member 14 can have several through-holes 44 along the length and/or width of the member 14 so long as the integrity of the member 14 is not affected.

In at least one embodiment, a therapeutic agent 30 is deposited on one surface of the member 14 that has an opening to the through-hole 44 as well as within the through-hole 44. In at least one embodiment, a first therapeutic agent 30 is deposited within the through hole 44 while a second therapeutic agent 30 is deposited on the surface of the member 14 where the opening to the through-hole 44 is positioned. In at least one embodiment, a first therapeutic agent 30 is deposited onto the first surface of the member 14 having the first opening of the through-hole 44 and at least partway into the passageway of the through-hole 44 and a second therapeutic agent 30 is deposited onto the second surface of the member 14 having the second opening and at least partway into the passageway of the through-hole 44. The first and second therapeutic agents 30 can be the same or different therapeutic agents 30. Similar to the V-shaped hole 46 discussed above, the through-hole 44 helps to keep the therapeutic agent 30, which is deposited on the surface of the member 14, engaged to the member 14.

It is within the scope of the invention for different coating retainers 22 to be combined to form a combination coating retainer 22. The combination coating retainer 22 in FIG. 12 is a non-limiting example of a combination coating retainer 22. In this embodiment, the coating retainer 22 is a combination of a channel 40 with two holes 44 extending from the bottom surface of the channel 40 at both ends of the channel 40. In this embodiment, the channel 40 extends across a portion of the width of the member 14, but it is within the scope of the invention for the channel 40 to extend across a portion of the length of the member 14. At the ends of the channel 40 are two holes 44 that extend further into the body of the member 14.

In this embodiment, the two holes 44 have the same depth, which is greater than the depth of the channel 40, but it is within the scope of the invention for the two holes 44 to have different depths, each of which are greater than the depth of the channel 40. It is also within the scope of the invention for a channel 40 to have one, two, three, four, five, six, seven, eight, nine, ten or more holes 44 along the length of the channel 40. In another aspect, this embodiment is a channel 40 with at least two depths along the length of the channel 40. In some embodiments, the holes 44 are at an oblique angle, similar to the blind holes of FIG. 9 discussed above. Note that as discussed in U.S. Pat. No. 6,709,379 to Brandau, the therapeutic agent 30 contained within the two holes 44 has a slower elution rate than the elution rate of the therapeutic agent 30 contained within the channel 40.

In at least one embodiment, layers of therapeutic agent 30 can be deposited within the combination coating retainer 22. In one embodiment, a first layer of therapeutic agent 30 is deposited within the two holes 44 as well as the bottom surface of the channel 40 and then a second layer of therapeutic agent 30 is deposited on top of the first layer of therapeutic agent 30. In one embodiment, the volume of the first therapeutic agent 30 is greater than the volume of the second therapeutic agent 30 partly due to the greater depth of the holes 44. In at least one embodiment, the therapeutic agent(s) deposited within the combination coating retainer 22 reduces delamination of the therapeutic agent 30 deposited onto the surface of the member 14.

The coating retainer 22 in FIG. 13 is another embodiment of a coating retainer 22 positioned within the body of the member 14. The coating retainer 22 can have any length. The body 43 of the coating retainer 22, positioned within the body of the member 14, can have any configuration, for example, but not limited to, a well 42, having a smaller length, or a channel 40, having a greater length. The coating retainer 22 illustrated in FIG. 13 has a body 43 with a variable width and an opening or hole 44 having a width smaller than the body 43 of the coating retainer 22. The smaller width of the opening or hole 44 helps to prevent delamination of the therapeutic agent 30 deposited within the body 43 of the coating retainer 22.

The embodiment in FIG. 13 has sides of the opening or hole 44 of the coating retainer 22 which are at an oblique angle to the surface of the member 14 and sides of the body 43 which are curvilinear, in contrast to the straight side of the channel 40 in FIG. 3. Thus, the width of the coating retainer 22 is smaller close to the surface of the member 14 and then becomes greater as the depth of the coating retainer 22 increases. It is within the scope of the invention for sides of the body 43 of the coating retainer 22 to be straight, instead of curvilinear.

If the body 43 of the coating retainer 22 is a well 42, therapeutic agent 30 deposited into the well 42 elutes from the well 42 through a hole 44. The size of the hole 44 affects the elution rate of the therapeutic agent 30 from the well 42. If the body 43 of the coating retainer 22 is a channel 40, therapeutic agent 30 deposited into the coating retainer 22 elutes from the channel 40 by at least one opening. The opening can be at least one hole 44 or one channel 40. The number and size of the holes 44 affects the elution rate of the therapeutic agent 30 from the body 43 of the coating retainer 22. In at least one embodiment, there are a plurality of holes 44 along the length of the coating retainer 22. Similarly, the width and length of the body 43 of the coating retainer 22 affects the elution rate of the therapeutic agent 30 from the coating retainer 22.

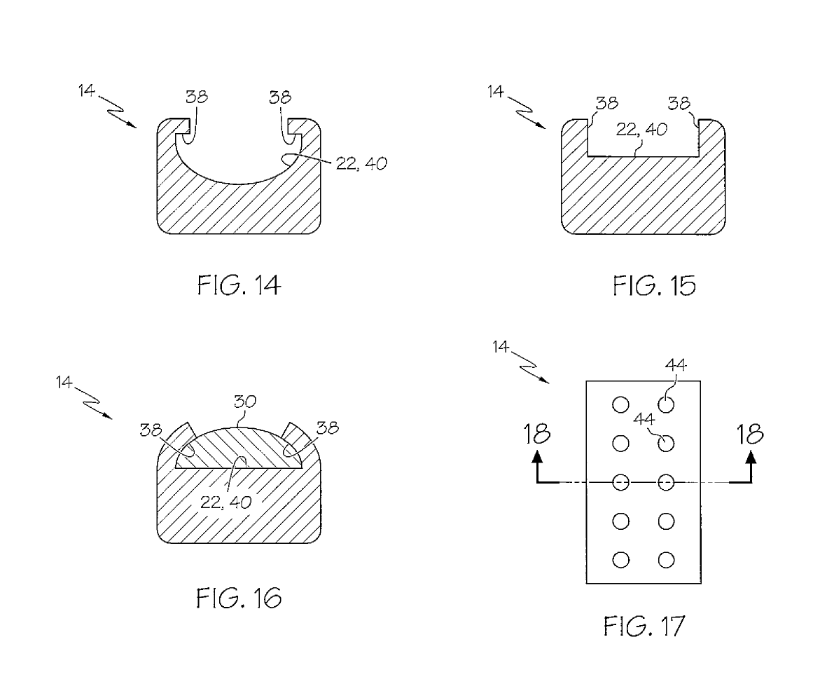

In at least one embodiment, the coating retainer 22 within the body of the member 14 has a means to protect a therapeutic agent 30 within the coating retainer 22, as shown, for example, in FIGS. 14-18. The coating retainer 22 in FIG. 14 is a channel 40 with lips 38. The lips 38 extend inwards over the therapeutic agent 30 deposited within the channel 40. It is within the scope of the invention for the sides and the bottom of the channel 40 to be curvilinear or straight, as illustrated for example by the channel 40 in FIG. 3. In an alternative embodiment, shown in FIGS. 15-16, the lips 38 are formed after the channel 40 is filled with the therapeutic agent(s) 30. Thus, the lips 38 have an unloaded state, shown in FIG. 15 and a loaded state, shown in FIG. 16. In this embodiment, the therapeutic agent(s) 30 is deposited into the channel 40 when the lips 38 are in the unloaded state. Once the therapeutic agent(s) 30 is deposited, the lips 38 are pressed inwards into a loaded state so that they extend over the therapeutic agent 30 thereby decreasing the delamination of the therapeutic agent(s) 30 within the channel 40.

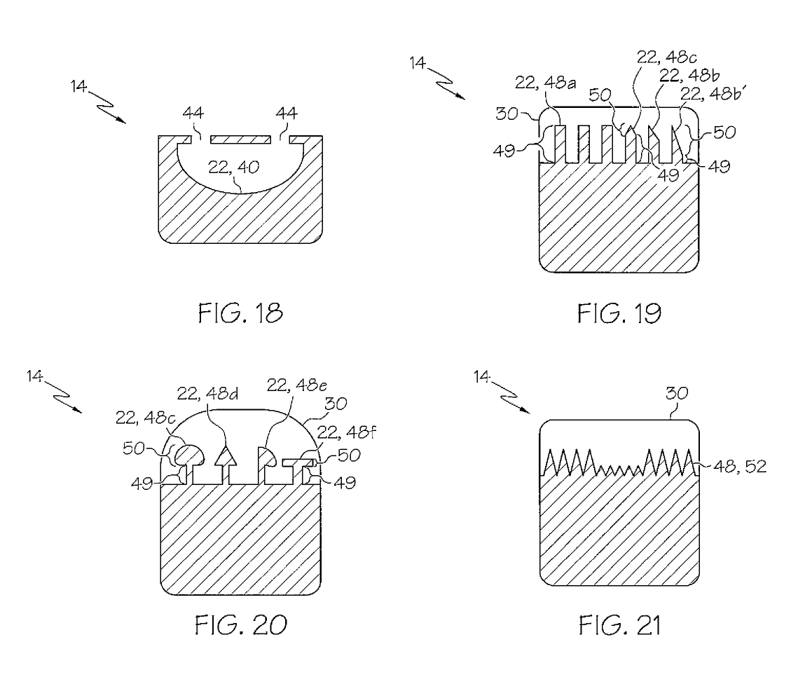

Another means by which the therapeutic agent(s) 30 in a coating retainer 22 can be protected is shown in FIGS. 17 and 18. FIG. 17 is a top view of the straight section 16 of member 14 with two rows of holes 44. Although the holes 44 have the same size, it is within the scope of the invention for the holes 44 to have any size and configuration. In addition, it is within the scope of the invention for the holes 44 to be in any pattern other than the two rows shown in FIG. 17. However, the size and position of the holes 44 should be chosen so that the portion(s) of the member 14 between the holes 44 is of a sufficient size to maintain the structural integrity of those portion(s) of the member 14, particularly after the therapeutic agent 30 has been eluted from the coating retainer 22.

FIG. 18 is a cross-section of the member 14 in FIG. 17 taken at line 18-18. As seen in FIG. 18, the holes 44 provide the means by which the therapeutic agent(s) 30 in the channel 40 is eluted from the channel 40. As discussed above, the size and number of the holes 44 affect the elution rate of the therapeutic agent 30 deposited in the coating retainer 22. In this embodiment, the channel 40 is formed within the body of the member 14. The channel 40 has a variable width, similar to the coating retainer 22 in FIG. 13. However, in this embodiment, the width of the channel 40 near the surface of the member 14/the holes 44 is greater the width of the bottom of the channel 40.

Coating retainers 22 engaged to the surface of one of the sides of the member 14 are shown in FIGS. 19-24. The depth of the therapeutic agent 30 deposited onto a member 14 with coating retainers 22 extending from the surface of the member 14 can be less than the height of the coating retainer 22, equal to the height of the coating retainer 22, or greater than the height of the coating retainer 22, i.e. overcoating. As discussed above, coating retainers 22 engaged to the surface of the member 14 may be positioned on at least one portion of the surface of the member 14 or on the entire surface of the member 14.

FIGS. 19-21 illustrate different coating retainers 22 that are protrusions 48 extending away from the surface of the member 14. In FIG. 19, the coating retainer 22 consists of a plurality of protrusions 48 extending from the surface of the member 14. In FIG. 19, the protrusions 48a have a substantially uniform width and height. However, it is within the scope of the invention for the body 49 of the protrusion 48 to have any shape, i.e. width and height, as shown, for example, by protrusions 48b, 48b′ and 48c. The protrusions 48 in FIGS. 19 and 20 comprise a body 49 and a cap region 50. The cap region 50 includes the upper surface of the protrusion 48. The cap region 50 can have the same configuration as the body 49, shown for example by protrusion 48a or the cap region 50 can have a different configuration than the body 49, shown for example by protrusion 48c. Protrusion 48a has a top surface that is horizontal to the surface of the member 14. This protrusion 48 embodiment can be described as being pillar shaped. It is also within the scope of the invention for the protrusion 48, body 49 and/or cap region 50, to have a variable width, in contrast to protrusion 48a. It at least one embodiment, the width of the protrusion 48 is greater near the surface of the member 14. In one embodiment, the protrusion 48 has the configuration of the pyramid, shown for example, in FIG. 21.

Note that the cap region 50 of the protrusion 48 can be any length, as shown, for example, by protrusions 48b and 48b′. The different configuration may be as simple as the top surface of the protrusion 48 being at an oblique angle to the surface of the member 14, like protrusion 48b in FIG. 19. Note that the cap region 50 of protrusion 48b′ has the same configuration as the cap region 50 of protrusion 48b but that the length of the cap region 50 of protrusion 48b′ is greater than the length of the cap region 50 of protrusion 48b. Alternatively, the cap region 50 may have a triangular shape with a peak, like an obelisk, as shown, for example, by protrusion 48c in FIG. 19. The widths of the cap regions 50 of protrusions 48b,c is at most equal to the width of the body 49 of the protrusion 48.

It is also within the scope of the invention for the protrusions 48 to have a cap region 50 where at least a portion of the cap region 50 has a width greater than the width of the body 49 of the protrusion 48, as shown in FIG. 20. Thus, in this embodiment, the width of the protrusion 48 is smallest near the surface of the member 14. As shown in FIG. 20, the cap region 50 can have a variety of configurations, for example, but not limited to, a mushroom-like cross-sectional shape 48c, an arrow-like cross-sectional shape 48d, a P-shaped cross-sectional shape 48e, or a T-shaped cross-sectional shape.

When a therapeutic agent 30 deposited onto a surface having protrusion(s) 48, the protrusion(s) 48 reduce delamination of the therapeutic agent 30 from the surface of the member 14. In at least one embodiment, when a therapeutic agent 30 is deposited onto a surface that has protrusions 48 with a cap 50, such as those illustrated, for example, in FIG. 20, the therapeutic agent 30 will be engaged to the protrusions 48c-f in the manner of a hook and loop material or VELCRO®. Methods of applying the therapeutic agent 30 to a member 14 with protrusions 48 are described in greater detail below.

The therapeutic agent 30 is deposited onto the surface of the member 14, where it settles between the protrusions 48, as discussed in greater detail below. Note that with this embodiment, a cross-section of the length of the member 14 would look the same as the cross-section of the width of the member 14, shown in FIG. 19. Although, the protrusions 48 illustrated in FIG. 19 are in rows along the length and width of the member 14, the protrusion 48 can be positioned on the surface of the member 14 in any design desired, e.g. random, zig-zag. The use of a mask in conjunction with chemical vapor deposition (CVD), physical vapor deposition (PVD), or pulsed laser deposition (PLD), to form protrusions 48 in any design desired is discussed in greater detail below.

In FIG. 21, the protrusion(s) 48 are made of metal oxide 52 which comprises peaks and valleys that retain therapeutic agents 30 on the surface of the member 14. Examples of metal oxide 52 that can be used include, but are not limited to, aluminum oxide, magnesium oxide, iron oxide, iridium oxide (Irox), iridium-iridium oxide (Ir—Irox), titanium oxide, titanium-iridium-iridium oxide (Ti—Ir—Irox), titanium-nitrogen oxide (TiNOx), titanium-titanium nitrogen oxide (Ti—TiNOx), tantalum oxide, tungsten oxide, and niobium oxide. In at least one embodiment, the member 14 is made of a metal which is then oxidized to form the protrusions 48 of metal oxide 52 on the surface of the member 14. It is within the scope of the invention for the member 14 to be made of a different material than the metal oxide 52. In at least one embodiment, the un-oxidized metal is deposited onto the surface of the member 14 and then oxidized. It is within the scope of the invention for the metal or metal oxide 52 to be deposited onto the surface of the member 14 in any suitable manner, for example, but not limited to chemical vapor deposition (CVD), physical vapor deposition (PVD) or pulsed laser deposition (PLD). In at least one embodiment, the metal or metal oxide 52 is deposited onto the surface of the member 14 by plasma deposition. In at least one embodiment, the metal oxide 52 is engaged to the surface of the member 14 by any suitable means.

The therapeutic agent 30 deposited onto the protrusions 48 of metal oxide 52 can have any depth. In at least one embodiment, the therapeutic agent 30 has a depth equal to the height of the protrusions 48 of metal oxide 52. In at least one embodiment, the therapeutic agent 30 has a depth greater than the protrusions 48 of metal oxide 52, i.e. overcoating, as shown in FIG. 21. In this embodiment, the therapeutic agent 30 in the valleys of the metal oxide 52 enhances the adhesion of the therapeutic agent 30 deposited over the protrusions 48 of metal oxide 52. Note that with this coating retainer 22 embodiment, a cross-section of the length of the member 14 would look the similar to a cross-section of the width of the member 14, shown in FIG. 21.

FIG. 22 illustrates a coating retainer 22 that is a channel 40 formed on the surface of the member 14. The edges 41 of the channel 40 are engaged to the surface of the member 14. The edges 41 form the four sides of the channel 40 and the surface of the member 14 is the bottom surface of the channel 40. In this embodiment, the edges 41 of the channel 40 help prevent the delamination of the therapeutic agent 30 deposited within the channel 40. In at least one embodiment, the edges 41 are made from the same material as the member 14. In at least one embodiment, the edges 41 are made from a different material than the member 14. In at least one embodiment, the edges 41 are protrusions 48. In some embodiments, the edges 41 are continuous. In other embodiments, the edges 41 are discontinuous. In this embodiment, the discontinuous edges 41 are formed of a plurality of protrusions 48, with a space between adjacent protrusions 48. Depending on the space between adjacent protrusions 48, therapeutic agent 30 deposited in the channel 40 can elute through the spaces between adjacent protrusions 48.

The edges 41 may be made from any suitable biocompatible materials including one or more polymers, one or more metals or combinations of polymer(s) and metal(s). Examples of suitable materials include biodegradable materials, and polymers such as polyester and polycarbonate copolymers. Examples of suitable biodegradable materials are listed above. Examples of suitable metals include, but are not limited to, stainless steel, titanium, tantalum, platinum, tungsten, gold and alloys or oxides of any of the above-mentioned metals. Examples of suitable alloys include platinum-iridium alloys, cobalt-chromium alloys including Elgiloy and Phynox, MP35N alloy and nickel-titanium alloys, for example, Nitinol.

FIGS. 23 and 24 illustrate another embodiment of coating retainer 22, a stainless steel shield 64, engaged to the surface of the member 14. FIG. 23 is a cross-section of a member 14 with the stainless steel shield 64 engaged thereto and FIG. 24 is a top view of the stainless steel shield 64 of FIG. 23. As shown in FIG. 23, the stainless steel shield 64 extends along the width of the member 14, but it is within the scope of the invention for the stainless steel shield 64 to extend along the length of the member 14.

In at least one embodiment, a stainless steel shield 64 has a therapeutic agent 30 electrocoated thereon. In at least one embodiment, the therapeutic agent 30 is electrocoated onto the entire outer surface of the stainless steel shield 64 before the stainless steel shield 64 is engaged to the member 14. In this embodiment, the therapeutic agent 30 positioned between the member 14 and the stainless steel shield 64 helps reduce delamination of the therapeutic agent 30 deposited on the other surfaces of the stainless steel shield 64. In at least one embodiment, the therapeutic agent 30 is electrocoated onto the outer surface of the stainless steel shield 64 after the stainless steel shield 64 has been engaged to the member 14.

In at least one embodiment, the stainless steel shield 64 has an elongated portion 66 and a transverse portion 68. In at least one embodiment, the stainless steel shield 64 also has a cap 70. In this embodiment, the cap 70 of the stainless steel shield 64, which extends over a portion of the therapeutic agent 30, helps reduce delamination of the therapeutic agent 30 deposited onto the outer surface of the stainless steel shield 64. Note that the cap 70 and transverse portion 68 may be two separate pieces that are engaged to one another by any suitable means or the transverse portion 68 and cap 70 may be manufactured as a single piece, e.g. as a rivet.

In some embodiments, the elongated portion 66 comprises two segments which are parallel to the surface of the member 14. Each of the two segments extends from either side of the transverse portion 68 to which they are engaged. Although the two segments in FIG. 23 have approximately the same length, it is within the scope of the invention for one segment have a greater length than the other segment.

In at least one embodiment, not shown, the elongated portion 66 is formed from a hollow tube with a therapeutic agent contained within the tube. In some embodiments, the hollow tube has one closed end and one open end. In other embodiments, the hollow tube has two closed ends. In some embodiments, the hollow tube has two open ends. In at least one embodiment, the hollow tube is a micro tube. It is within the scope of the invention for the hollow tube to have any cross-sectional configuration. In some embodiments, the hollow tube has an oval shaped cross-section. In other embodiments, the hollow tube has a round shaped cross-section before the therapeutic agent is deposited into the tube and then the tube is compressed so that it has an oval shaped cross-section. Materials that can be used to make the tube include, but are not limited to, stainless steel, polymers, and biodegradable materials.

In at least one embodiment, the transverse portion 68, which is perpendicular to the elongated portion 66, has a length greater than the thickness of the elongated portion 66 so that it extends beyond the top and bottom surfaces of the segments of the elongated portion 66. In some embodiments, one end of the transverse portion 68 is engaged to the surface of the member 14, as shown by the solid lines in FIG. 23. In other embodiments, the transverse portion 68 extends into the member 14. In one embodiment, the transverse portion 68 is in the form of a rivet that extends through the coated elongated portion 66,30 and from one side of the member 14 to the opposite side of the member 14, shown in FIG. 23 by dashed lines extending through the member 14. In at least one embodiment, the stainless steel shield 64 is engaged to a member 14 manufactured of polymer. In one embodiment, the elongated portion 66 and the transverse portion 68 are formed as one piece, therefore the two segments of the elongated portion 66 are not engaged to the transverse portion 68. In one embodiment, the stainless steel shield 64 does not have a transverse portion 68, instead it only has an elongated portion 66.

Similar to the channels 40, the stainless steel shield 64 has a length. The length of the stainless steel shield 64 ranges from at least equal to a quarter of the length or width of the member 14 to the entire length or width of the member 14. In at least one embodiment, when the stainless steel shield 64 extends across the width of the member 14, the width of the stainless steel shield 64 is at most equal to the width of the member 14. In FIG. 23, the stainless steel shield 64 has a width slightly less than the width of the member 14 so that when the therapeutic agent 30 is applied to the stainless steel shield 64, the width of the stainless steel shield 64 with the therapeutic agent 30 is substantially the same as the width of the member 14. In one embodiment, the width of the coated stainless steel shield 64 ranges from at least one quarter the width of the member 14 to substantially the same width as the member 14.

In at least one embodiment, the stainless steel shield 64 has a length slightly less than the length of the member 14. In one embodiment, the length of the coated stainless steel shield 64 ranges from at least one quarter the length of the member 14 to substantially the same length as the member 14. As shown in FIG. 24, it is within the scope of the invention for the stainless steel shield 64 the have more than one transverse portion 68 along the length of the stainless steel shield 64 and it is within the scope of the invention for the stainless steel shield 64 to have at least one hole 44 extending through the stainless steel shield 64. As shown in FIG. 24, the stainless steel shield 64 has two transverse portions 68 and three holes 44 along the length of the stainless steel shield 64. In some embodiments, therapeutic agent 30 within an elongated portion 66 made from a hollow tube is eluted through the holes 44.

In at least one embodiment, the elongated portion 66 with the therapeutic agent 30 applied thereto is engaged to the member 14 by transverse portion 68 in the following manner. First, a hole is drilled through the coated elongated portion 66,30 and a small distance into the member 14. In at least one embodiment, the hole is made by focused ion beam (FIB) sputtering. The use of focused ion beam technology in fabrication is described in T. Tanaka et al., “Micrometer-scale fabrication and assembly using focused ion beam,” Thin Solid Films, 509 (2006) 113-117, hereby incorporated by reference in its entirety. In some embodiments, the hole is about 1 micrometer to about 20 micrometers in size. Then the transverse portion 68 is inserted into the hole. In some embodiments, the transverse portion 68 is silicon oxide (SiO) pin. In other embodiments, the transverse portion 68 is a rivet. In at least one embodiment, the transverse portion 68 is fabricated using focused ion beam technology.

In at least one embodiment, the stainless steel shield 64 is held onto the surface of the member 14 by lips 38 of a channel 40. Thus, this embodiment is a combination coating retainer 22 in which the coating retainer 22 of FIGS. 15/16 and the coating retainer 22 of FIGS. 23/24 are combined. In this embodiment, the elongated portion 66 with the therapeutic agent 30 applied thereto of FIG. 23 is placed in the channel 40 of the coating retainer 22 of FIG. 15. Then the lips 38 of the channel 40 extended inwards over the coated elongated portion 66,30, similar to the lips 38 extending inwards over the therapeutic agent 30, as shown in FIG. 16. Note that in this embodiment, the lips 38 keep the stainless steel shield 64 in the channel 40. In at least one embodiment, the stainless steel shield 64 in the channel 40 has at least one hole 44 along the length of the stainless steel shield 64. In some embodiments, the stainless steel shield 64 does not have any transverse portion(s) 68.

Another type of coating retainer 22 is a porous material 34 which can hold a therapeutic agent 30. FIGS. 25-29 show different embodiments where a porous material 34 used as a coating retainer 22. Any porous material/substance may be used for the porous material 34. Examples of porous material 34 include but are not limited to sintered metal, porous ceramics (sintered or chemically created) and porous polymeric surfaces. In at least one embodiment, the porous material 34 is a reservoir for a therapeutic agent 30.

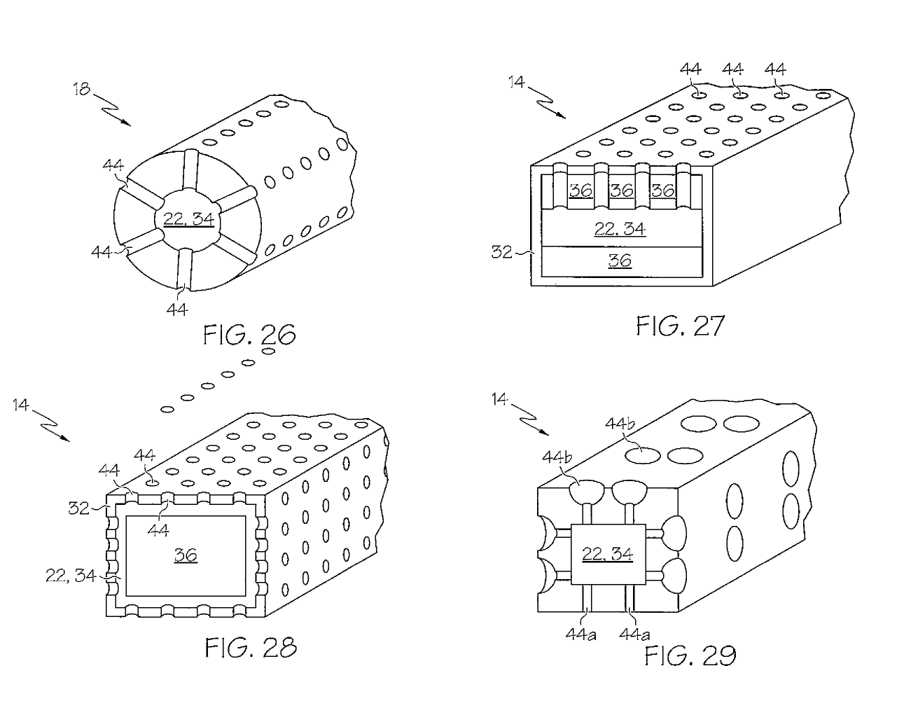

In at least one embodiment, the porous material 34 forms the core of the member 14, as shown in FIG. 25, Note that member 14 may be manufactured of wire 18 made of porous material 34, as shown in FIG. 26. In at least one embodiment, the porous material 34 is sandwiched between two sections of stent material 36, as shown in FIG. 27. Stent material 36 is any material that can be used to fabricate a stent 10, examples of which are listed below. In at least one embodiment, the porous material 34 surrounds a core of stent material 36, as shown in FIG. 28. In these embodiments, holes 44 go from the surface of the member 14 to the porous material 34, thereby providing a channel or passageway for the therapeutic agent 30 to elute/diffuse from the porous material 34 to the surface of the member 14. In at least one embodiment, the holes 44 are created by laser cutting. In at least one embodiment, there is a coating 32 around the circumference of the member 14, such as is illustrated in FIGS. 27 and 28. Materials used for the coating 32 include but are not limited to polymers, stent material 36 and any combination thereof. Note that the holes 44 also go through the coating 32.

Although the holes 44 shown in FIGS. 25-28 are substantially round with substantially uniform size, as discussed above, the holes 44 can have any shape and size. In at least one embodiment, the hole 44 is a channel 40, as shown, for example, in FIG. 25. In at least one embodiment, the holes 44 have at least one diameter. For example, some of the holes 44 in FIG. 29 have a funnel shape where the hole 44 has a wide opening at the surface of the member 14 and a narrower channel that goes to the porous material 34. In at least one embodiment, the holes 44a on one side of the member 14 are larger than the holes 44b on the adjacent side of the member 14, as seen for example in FIG. 25. In at least one embodiment, the member 14 has holes 44 leading to the porous material 34 which are at least two shapes/sizes. This is shown, for example, in FIG. 29 where one side of the member 14 has one hole 44a design while the other sides of the member 14 have a second hole 44b design, a funnel shaped design. In addition the holes 44 can be in any distribution or pattern, other than the series of rows shown in FIGS. 25-29. In FIG. 26, the holes 44 are radially situated.

The shape, size and distribution of the holes 44 affect the rate the therapeutic agent 30 elutes/diffuses from the porous material 34. For example, a larger hole 44 allows more of the therapeutic agent 30 to elute from the porous material 34 than a smaller hole 44. Similarly, a higher distribution of holes 44 on the surface of the member 14/wire 18 will target more therapeutic agent 30 to a particular location than will a lower distribution of holes 44. Thus, if no therapeutic agent 30 is to be eluted from a particular side or area of the member 14/wire 18, that particular side or area of the member 14 can be made without any holes 44, thereby preventing the elution/diffusion of the therapeutic agent 30 to that area. This is illustrated, for example, in FIG. 27, where only one side of the member 14 has holes 44.

In at least one embodiment, the member 14 has a second type of coating retainer 22 on the side of the member 14 that does not have holes 44. In this embodiment, the member 14 has a core of porous material 34 that elutes therapeutic agent 30 from one side of the member 14 and a coating retainer 22, e.g. a clamp 54 or a pin 58, on another side of the member 14. In at least one embodiment, only a portion of the member 14 has porous material 34. For example, the body of the proximal section of the member 14 can have a section of porous material 34 while the distal section of the member 14 does not have a section of porous material 34. In one embodiment, a second type of coating retainer 22, e.g. channel 40, is positioned in the distal section of the member 14. In this embodiment, therapeutic agent 30 is eluted from the porous material 30 in the proximal section of the member 14 and therapeutic agent 30 is eluted from the channel 40 in the distal section of the member 14. In this embodiment, the channel 40 can be on the same side of the member 14 as the holes 44 extending from the porous material 34, or the channel 40 can be on a different side of the member 14 than the holes 44.

In at least one embodiment, the holes 44 contain a substance that facilitates the elution/diffusion of the therapeutic agent 30 after the stent 10 has been placed in a lumen. In at least one embodiment, a substance that inhibits the elution/diffusion of the therapeutic agent 30 is deposited into the holes 44. In these embodiments, the therapeutic agent 30 does not elute from the stent 10 until it is placed in a body lumen. In at least one embodiment, the holes 44 are blocked by a substance. In some embodiments, the substance blocking the holes 44 is a coating 32, deposited onto the member 14 after the holes 44 have been created. In some embodiments, the coating 32 is made of biodegradable material. In this embodiment, the therapeutic agent 30 is eluted from the porous material 34 after the holes 44 are opened due to the degradation of the biodegradable coating 32. In at least one embodiment, the porous material 34 also contains a substrate which causes the therapeutic agent 30 to elute in a controlled manner. In at least one embodiment, the substrate works in conjunction with a carrier or binding agent.

Examples of a substance that inhibits the elution/diffusion of a therapeutic agent 30 includes, but is not limited to, biodegradable materials, materials that can be enzymatically degraded, materials that can be degraded as a result of response stimulated release of either enzymes or other agents, such as hydrogen peroxide that is released by macrophages as part of the inflammatory response, thermo-responsive polymers, water swelling polymers and surfaces that bind specific antigens or antibodies which results in a change in permeability. Examples of suitable materials for the biodegradable material are listed above.

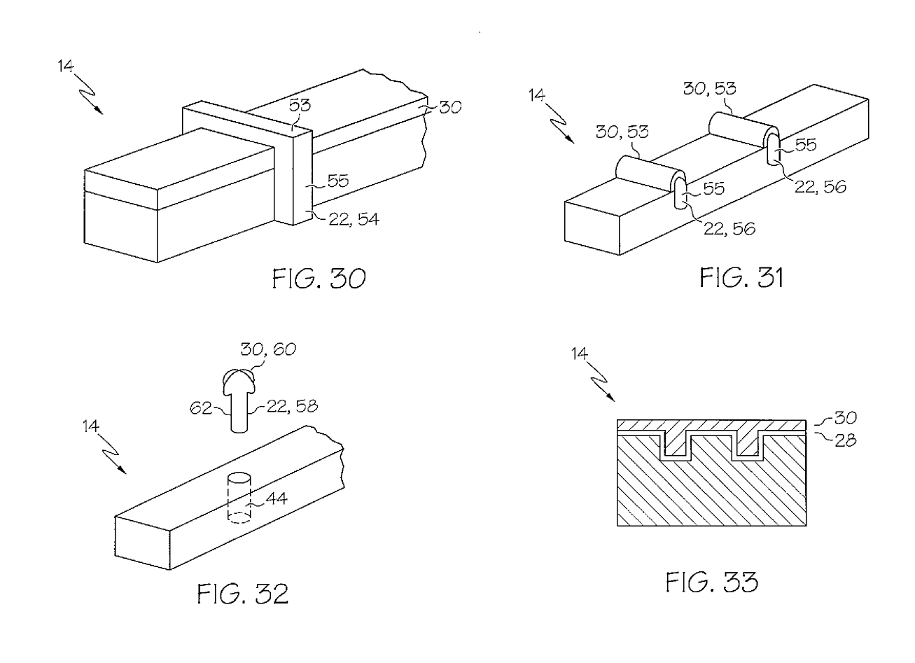

Other coating retainers 22 which can be used to deliver a therapeutic agent 30 from a stent 10 include a clamp 54/staple 56, and a pin 58. As shown in FIGS. 30 and 31, the clamp 54 and the staple 56 have the same configuration, i.e. a body 53 with two arms 55. Thus, a clamp 54 and a staple 56 are the same coating retainer 22 used to reduce delamination of a therapeutic agent 30 in different ways, discussed in greater detail below.

The body 53 of the clamp 54/staple 56 has a width greater than the width of the member 14 so that the arms 55, which are on both ends of the body 53, engage opposite sides of the member 14. The arms 55 can have any length so long as they engage the clamp 54/staple 56 to the member 14. In addition, the arms 55 and body 53 can have any thickness. In at least one embodiment, the clamp 54/staple 56 is made from a biocompatible material that does not degrade. In at least one embodiment, the clamp 54/staple 56 is made from a biodegradable material.

The clamp 54 reduces delamination of a therapeutic agent 30 deposited onto the surface of a member 14 by overlaying the therapeutic agent 30, i.e. “clamping” the therapeutic agent 30 onto the member 14. In contrast, the staple 56 reduces delamination of a therapeutic agent 30, which is deposited onto the body 53 of the staple 56. Note that the therapeutic agent 30 can be deposited about the entire surface of the body 53 of the staple 56 or only a portion of the body 53 of the staple 56. In at least one embodiment, therapeutic agent 30 deposited onto the underside of the body 53 of the staple 56 prevents delamination of the therapeutic agent 30 deposited onto the other surfaces of the staple 56.

FIG. 32 shows a coating retainer 22 which is a pin 58. The pin 58 has a head 60 and a shaft 62. The head 60 of the pin 58 has at least one therapeutic agent 30 deposited thereon. The pin 58 is engaged to the member 14 by inserting the shaft 62 of the pin 58 into a hole 44 in the member 14. The head 60 can have any shape and size, depending upon the amount of therapeutic agent 30 to be deposited onto the pin 58. The cap regions 50 of the protrusions 48 in FIG. 20 are some non-limiting examples of configurations that the head 60 of the pin 58 can have. The diameter of the head 60 is at a minimum equal to the diameter of the shaft 62. Although the shaft 62 of the pin 58 in FIG. 32 has a round shape, the shaft 62 can have any shape desired. The shape of the hole 44 is complementary to the shape of the shaft 52. In at least one embodiment, the pin 58 has a thumbtack shape. In at least one embodiment, the pin 58 has a pushpin shape. In at least one embodiment, the therapeutic agent 30 deposited onto the pin 58 surrounds the head 60 and the upper portion of the shaft 62.

Note that if the hole 44 into which the shaft 62 of the pin 58 is inserted into has a depth less than the length of the shaft 62, a plurality of pins 58 form a coating retainer 22 that is similar to the protrusions 48 shown in FIG. 20, with the difference that at least a portion of the shaft 62 is positioned within the body of the member 14. In at least one embodiment, the depth of the hole 44 is less than the length of the shaft 62 so that there is a distance between head 60 of the pin 58 and the surface of the member 14. In at least one embodiment, the depth of the hole 44 equal to the length of the shaft 62 of the pin 58. If the hole 44 is equal to the length of the shaft 62, the bottom surface of the head 60 of the pin 58 abuts the surface of the member 14.

In at least one embodiment, the depth of the hole 44 is greater than the length of the shaft 62 of the pin 58. In one embodiment, the hole 44 is a through-hole extending from one surface of the member 14 to the opposite surface of the member 14. In this embodiment, after the pin 58 is placed into the hole 44 on one surface of the member 14, the hole 44 on the opposite surface of the member 14 becomes a blind hole 44, which are discussed above. Thus, a therapeutic agent 30 can be deposited onto the head 60 of the pin 58 on the first surface and on the opposite surface of the member 14, a therapeutic agent 30 can be deposited at least into the hole 44. Therapeutic agent 30 can also be deposited onto the opposite surface, with the therapeutic agent 30 deposited within the hole 44 reducing delamination of the therapeutic agent 30 on the surface, as discussed above.

The above discussion about the different coating retainers 22 and combinations of coating retainers 22 on a member 14 is intended to be illustrative and not exhaustive. This description will suggest many variations and alternatives to one of ordinary skill in this art. The various elements shown in the individual figures and described above may be combined or modified for combination as desired.

The invention also directed to methods of manufacturing some of the coating retainers 22 described herein. In some embodiments, a laser is used to form the coating retainers 22. In other embodiments, ion beam etching is used to form the coating retainers 22. Coating retainers 22 that can be made by a laser or ion beam etching include, but are not limited to, channels 40, wells 42, holes 44, V-shaped holes 46, and protrusions 48. For the coating retainers 22 positioned within the body of the member 14, e.g. channels 40, wells 42, holes 44 and V-shaped holes 46, the laser removes a portion of the body to form the coating retainer 22.

In at least one embodiment, an energy source is used to make at least one indentation in at least one surface of at least one member 14 of a bare metal stent 10. In at least one embodiment, the at least one surface is the abluminal surface of the member 14. Examples of energy sources that can be used include, but are not limited to, a laser or ion beam, and plasma ion impingement implantation. Examples of indentations include but are not limited to holes 44, channels 40, and irregular structures. In at least one embodiment, the indentations have a depth less than ⅓ of the thickness of the member 14. In at least one embodiment, the channels 40 are at an oblique angle to the longitudinal axis of the member 14. As discussed above, the indentations can have any pattern or configuration.

In at least one embodiment, after the indentations have been made in the surface of the member 14, a passivation layer 28 is deposited onto the surface of the member 14 to re-passivate the member 14. In at least one embodiment, the passivation layer 28 reduces corrosion. In at least one embodiment, the passivation layer 28 on the member 14 is formed by depositing a layer of chromium onto the surface of the member 14 with an acid. In at least one embodiment, the passivation layer 28 is a layer of iridium oxide (Irox).

After the addition of the passivation layer 28, the member 14 is coated with metal and/or metal oxides using for example, but not limited to, chemical vapor deposition (CVD), physical vapor deposition (PVD) or pulsed laser deposition (PLD). Subsequently, the member 14 is coated with a therapeutic agent 30. The therapeutic agent 30 may be deposited onto the member 14 by spray-coating, dip-coating, roll-coating, print-coating or any other suitable method. FIG. 33 is a cross-section of a member 14 that has undergone the process just described. Note that the layer of therapeutic agent 30 can have any thickness.

In at least one embodiment, a mask and a chemical etching process is used to remove at least one portion of the surface of the member 14 to form at least one indentation. The mask can have any design. In at least one embodiment, the mask design is chosen so that the resulting textured surface acts as a healing promoter during and/or after drug elution. Any suitable chemical etching method may be used. An example of a textured surface that can be formed by this method is illustrated in FIG. 34. In this embodiment, the entire stent 10 or only a portion of the stent 10 can undergo this process. Thus, some members 14 of the stent 10 will have indentations while other members 14 will have no indentations. In at least one embodiment, core shell particles 100 are deposited onto the textured surface of the member 14, as illustrated in FIG. 35. Core shell particles 100 are discussed in greater detail below in reference to FIG. 45.

In at least one embodiment, laser or ion beam processing is used to remove at least a portion of the surface of the member 14 to form at least one indentation. In this embodiment, no mask is used. In at least one embodiment, the design etched by the laser or ion beam is chosen so that the resulting textured surface acts as a healing promoter during and/or after drug elution. An example of a textured surface that can be formed by this method is illustrated in FIG. 34. In this embodiment, the entire stent 10 or only a portion of the stent 10 can undergo this process. Thus, some members 14 of the stunt 10 will have indentations while other members 14 will have no indentations.

In at least one embodiment, a mask and deposition of a metal or metal oxide 106 is used to create a textured surface on the member 14. The mask can have any design. In at least one embodiment, the metal/metal oxide 106 is deposited directly onto the surface of the member 14. In one embodiment, the member 14 is bare metal, i.e. a bare metal stent 10. In at least one embodiment the metal/metal oxide 106 is deposited onto an intermediate layer 104 that has been deposited onto the member 14. In one embodiment, the member 14 is bare metal, i.e. a bare metal stunt 10. An example of a textured surface that can be formed by this method is illustrated in FIG. 36. As shown in FIG. 36 the metal/metal oxide 106 is deposited onto an intermediate layer 104 to form a textured surface. In this embodiment, the entire stent 10 or only a portion of the stent 10 can undergo this process. Thus, some members 14 of the stent 10 will have indentations while other members 14 will have no indentations. FIG. 37 is the member of FIG. 36 with core shell particles 100 deposited into the indentations of the textured surface of the member 14.

Examples of suitable metals and metal oxides that can be used to create a textured surface on the member 14 include, but are not limited to, aluminum, aluminum oxide, magnesium oxide, iron oxide, iridium, iridium oxide (Irox), iridium-iridium oxide (Ir—Irox), titanium, titanium oxide, titanium-iridium-iridium oxide (Ti—Ir—Irox), titanium-nitrogen oxide (TiNOx), titanium-titanium nitrogen oxide (Ti—TiNOx), tantalum, tantalum oxide, tungsten, tungsten oxide, niobium, niobium oxide, gold, and platinum. Examples of suitable materials for the intermediate layer 104 include, but are not limited to, titanium and iridium. It is within the scope of the invention for the material used for the intermediate layer 104 to be the same or different than the metal/metal oxide 106.

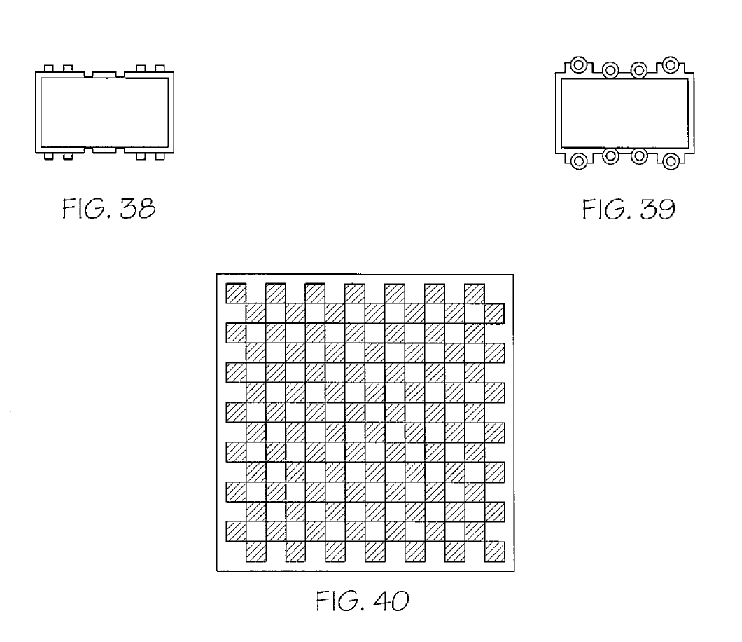

In at least one embodiment, a textured surface on the member 14 is formed by first depositing metal or metal oxide onto a member 14 using a mask to form a textured surface, as described above, and then using a mask and chemical etching to remove at least one portion of the textured surface of the member 14, as described above. An example of a textured surface that can be formed by this method is illustrated in FIG. 38. As shown in FIG. 38, the metal/metal oxide 106 is deposited onto an intermediate layer 104. Also shown in FIG. 38, the chemical etching process removes at least one portion of the intermediate layer 104. In FIG. 38, both processes were done on the member 14. However, it is within the scope of the invention for one process (deposition of metal/metal oxide) to be done on one portion of the stent 10 and the second process (chemical etching with a mask) to be done on another portion of the stent 10. FIG. 39 shows the cross-section of the member 14 in FIG. 38 with core shell particles 100 deposited onto the textured surface of the member 14.

In at least one embodiment, a textured surface on the member 14 is formed by first depositing metal/metal oxide 106 onto a member 14 using a mask to form a textured surface, as described above, and then using a laser or ion beam treatment to remove at least a portion of the textured surface of the member 14. An example of a textured surface that can be formed by this method is illustrated in FIG. 38. As shown in FIG. 38, the metal/metal oxide 106 is deposited onto an intermediate layer 104. Also shown in FIG. 38, the laser or ion beam removes at least one portion of the intermediate layer 104. In FIG. 38, both processes were done on the member 14. However, it is within the scope of the invention for one process (deposition of the metal/metal oxide) to be done on one portion of the stent 10 and the second process (laser/ion beam treatment) to be done on another portion of the stent 10.

In at least one embodiment, the surface of a pre-cut stent tube can be stamped so that the members 14 of the stent will have an uneven surface. The therapeutic agent 30 deposited into depressions made in the surface of the member by the stamping process enhances the adhesion of the therapeutic agent 30 on the surface of the member 14. In at least one embodiment, a laser is used to create a pattern on the surface of the member 14, thereby causing the member 14 to have an uneven surface.

In at least one embodiment, a laser is used for form coating retainers 22 that protrude from the surface of the member 14, e.g. protrusions. To form coating retainers 22 that protrude from the surface of the member 14, the laser removes the portions of the body of the member 14 around the coating retainer 22. For example, to form the protrusions 48, the laser removes portions of the body of the member 14 surrounding the protrusions 48. In this embodiment, the member 14 has an original thickness that is greater than the thickness of the member 14 after the laser has made the protrusions 48. Thus, the original thickness is equal to the thickness of the member 14 and the height of the protrusions 48.

As mentioned above, protrusions 48 may be formed on the surface of a stent member 14 by using a mask in conjunction with chemical vapor deposition (CVD), physical vapor deposition (PVD) or pulsed laser deposition (PLD). The mask can have any design desired. Note that the position of the opening(s) in the mask determine the position of the protrusion(s) 48. In at least one embodiment, the protrusions 48 made by this method form at least one channel 48 on the surface of the member 14, shown for example in FIG. 22.

In at least one embodiment, a metal or a metal oxide is deposited onto the surface of the member 14 to form the protrusions 48. It is within the scope of the invention for the metal or metal oxide to be deposited directly onto a member 14 of a bare metal stent or for the metal or metal oxide to be deposited on an interlayer that will then be positioned between the bare metal stent and the protrusion 48. Examples of metals and metal oxides that may be used include, but are not limited to, aluminum, aluminum oxide, magnesium oxide, iron oxide, iridium, iridium oxide (Irox), iridium-iridium oxide (h-Irox), titanium, titanium oxide, titanium-iridium-iridium oxide (Ti—Ir—Irox), titanium-nitrogen oxide (TiNOx), titanium-titanium nitrogen oxide (Ti—TiNOx), tantalum, tantalum oxide, tungsten, tungsten oxide, niobium, niobium oxide, gold, and platinum.

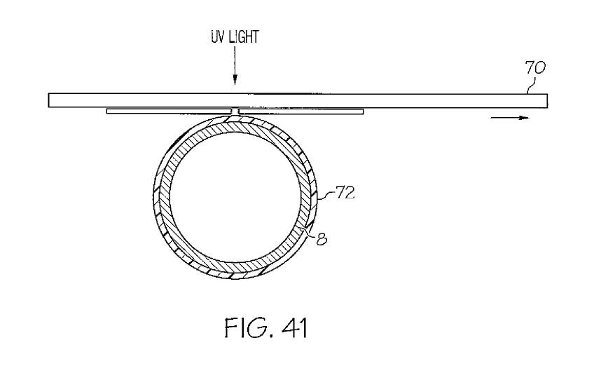

In at least one embodiment, channels 40 or wells 42, shown in FIG. 8, are made by a cylindrical lithography procedure which is illustrated in FIGS. 40-44. The cylindrical lithography procedure described herein is done to a stent tube 8 before the stent pattern is cut into the stent tube 8, however, the cylindrical lithography procedure can also be done on the finished cut stent 10. FIG. 40 shows an example of a mask design on a transparent glass plate 70 that can be used in the cylindrical lithography procedure. The mask design in FIG. 40 is a checkerboard design of clear and black areas, but any design can be used in the procedure. FIG. 41 is an end view of a stent tube 8 undergoing the cylindrical lithography procedure.

As shown in FIG. 41, the stent tube 8 has a coating of photoresist film 72. Ultraviolet light (UV) is directed towards the assembly, as indicated by the arrow in FIG. 41. The clear areas of the transparent glass plate 70 allow the UV light to pass through to the photoresist film 72. In negative resist, when the photoresist film 72 is exposed to the UV light, it hardens/becomes less soluble and remains engaged to the stent tube 8 while the portions of the photoresist film 72 that are not exposed to the UV light, i.e. the areas underneath the black areas of the transparent glass plate 70 can be removed from the stent tube 8 when the stent tube 8 is chemically processed. Chemical process steps can include a post bake step followed by a solvent wash step to remove un-irradiated photoresist. In positive resist, when the photoresist film 72 is exposed to the UV light, it becomes more soluble to the developer chemicals. Thus, you get a replica of your mask in the photoresist film 72. Although, both types of polymer photoresist can be used in this cylindrical lithography procedure, the discussion will focus on negative resist.

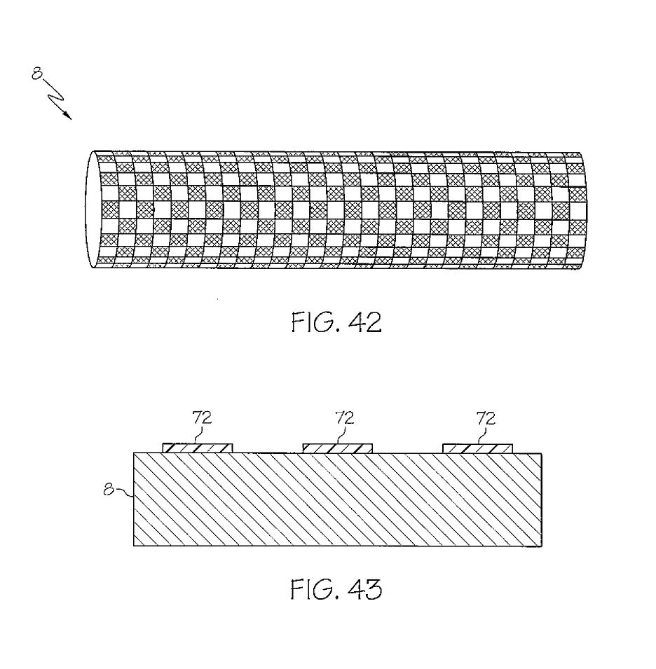

FIG. 42 shows how the stent tube 8 looks after the areas of photoresist film 72 that were not exposed to UV light are removed from the stent tube 8. Thus, the checkerboard design of the mask has been transferred to the stent tube 8 due to the continued engagement of squares of photoresist film 72 after the stent tube 8 has been chemically processed. In some embodiments, a laser direct writes a design onto the photoresist film 72, instead of using the cylindrical lithography procedure. In other embodiments, an electron beam direct writes a design onto the photoresist film 72, instead of using the cylindrical lithography procedure. Once the design is transferred to the photoresist film 72, the subsequent steps are the same as the subsequent steps described below that are followed in the cylindrical lithography procedure.

As stated above, the mask can have any design, thus the photoresist film 72 that remains engaged to the stent tube 8 can have the shape of a triangle, square, hexagon, circle, and irregular shapes. These shapes can have a size ranging from a submicron up to millimeters. In at least one embodiment, the photoresist film 72 is a line that is wavy, has a saw-tooth shape or other irregular lines. In one embodiment, the lines of photoresist film 72 follow the shape of the stent architecture.

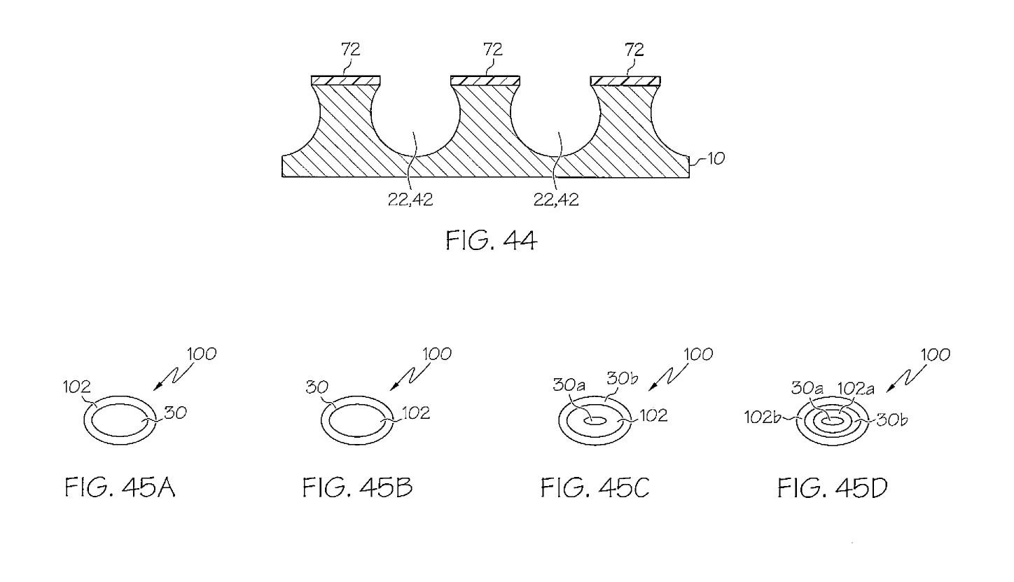

FIG. 43 is a cross-section of the stent tube 8 showing the areas of photoresist film 72 that are engaged to the surface of the stent tube 8. At this point, the stent tube 8 is ready to be laser cut into a stent 10. In at least one embodiment, after the stent 10 has been cut, it is electropolished. In at least one embodiment, after the stent 10 has been cut, it is etched by ion beams. The electropolishing procedure and the ion beam etching create under cut features, such as wells 42 in the surface of the stent 10. The depth of the under-cutting, and therefore the extent of the etching that occurs, depends on the parameters of the electropolishing or ion beam etching. FIG. 44 is the cross-section of FIG. 43 after the electropolishing procedure. The next step is to remove the remaining pieces of photoresist film 72 from the stent 10 by dissolving the photoresist film 72 with a solvent or by pyrolysis. After the remaining pieces of photoresist film 72 are removed, the surface of the stent 10 will look like the surface of the cross-section of the member 14 in FIG. 3. Note that because this procedure uses a stent tube 8 the entire stent 10 will have under cut areas, not just the straight portions of the members 14. In some embodiments, the stent 10 undergoes a passivation step after the electropolishing procedure.

Another means by which coating retainers 22 can be created on the surface of the stent 10 is by grit blasting. Grit blasting causes the surface of the stent 10 to become roughened/uneven. In this method, the stent 10 is placed on a mandrel and then grit is directed to the desired surface of the stent 10 so that it becomes uneven, thereby producing coating retainers 22 on the entire surface of the stent 10, not just the straight portions of the member 14. In at least one embodiment, the grit is only directed to the abluminal side of the stent 10. In at least one embodiment, ion beam etching or sputtering is used instead of grit blasting to cause the surface of the stent 10 to become roughened/uneven.

As mentioned above, there are several methods by which a therapeutic agent 30 can be applied to the member 14 so that it is deposited into/onto a coating retainer 22. In at least one embodiment, the therapeutic agent 30 is encapsulated in microbeads, which are deposited into/onto the coating retainer 22. Microbeads of therapeutic agents 30 can be used, for example, in the channel 40 illustrated in FIG. 3. In at least one embodiment, the therapeutic agent 30 is deposited into/onto the coating retainer 22 using a micro “foam in place” method. In this embodiment, the therapeutic agent 30 is the form of a solution, suspension, liquid or solid and after the therapeutic agent 30 is deposited onto the coating retainer 22 a foam is formed. In some embodiments, the foam formation is due to the reaction of components in the application. In other embodiments, the foam formation is due to the release of solvent. In at least one embodiment, the foam formation is due to an existing foam structure.

In at least one embodiment, therapeutic agent 30 is forms a part of a core shell particle 100, which is deposited into/onto a coating retainer 22. In at least one embodiment, core shell particles 100 allow for the controlled release of therapeutic agent 30 over time. The formation of core shell particles 100 is described in greater detail in commonly assigned US Patent Application Publ. No. 2006/0045901, entitled Stents With Drug Eluting Coatings, hereby incorporated by reference in its entirety. In at least one embodiment, the core shell particles 100 are 1 nm to 999 nm in diameter. In at least one embodiment, the core shell particles 100 are between 1 to 50 μm in diameter.

The core shell particles 100 can have any configuration desired. Some non-limiting possible configurations of core shell particles 100 are shown in FIGS. 45A-D. The invention contemplates other core shell particle 100 configurations. As shown in FIGS. 45A-D, the core shell particle 100 has at least one section/layer of therapeutic agent 30 and at least one section/layer of biodegradable material 102. Examples of suitable materials for the biodegradable layer 102 are listed above and discussed in US Patent Application Publ. No. 2006/0045901, entitled Stents With Drug Eluting Coatings. In at least one embodiment, the core of the core shell particle 100 is composed of biodegradable layer 102 material which is surrounded by a layer of therapeutic agent 30.

It is within the scope of the invention for the core shell particle 100 to have two, three, four, five, six, seven, eight, nine, ten or more layers 30,102. Note that the amount of material (therapeutic agent 30 or biodegradable material 201) forming each section/layer of the core shell particle 100 can be the same or different.

FIGS. 45A and B illustrate two different core shell particle 100 configurations. Each core shell particle 100 configuration comprises one layer of therapeutic agent 30 and one biodegradable layer 102. The biodegradable layer 102 can surround the therapeutic agent 30, as shown in FIG. 45A, or the biodegradable layer 102 can be surrounded by the therapeutic agent 30, as shown in FIG. 45B. When the biodegradable layer 102 surrounds a layer of therapeutic agent 30, degradation of the biodegradable layer 102 allows the elution of the therapeutic agent 30 from the core shell particle 100. Thus, the elution of the therapeutic agent 30 is delayed by the time it takes for the bioabsorbable layer 102 to degrade.

In at least one embodiment, the core shell particle 100 has several sections/layers of therapeutic agent 30, as illustrated in FIG. 45C-D. It is within the scope of the invention for the sections of therapeutic agents 30 of the core shell particle 100 to be different therapeutic agents 30. This allows the sequential delivery of different therapeutic agents 30. It is also within the scope of the invention for the different sections of therapeutic agent 30 to be the same therapeutic agent 30 but to have different concentrations. Thus, the design of the core shell particle 100 will depend upon the therapeutic regimen desired.

In at least one embodiment, the core shell particle comprises three layers 30/102. In one embodiment, the three layers of the core shell particle 100 comprises one biodegradable layer positioned between two layers of therapeutic agent 30a,b as illustrated in FIG. 45C. Alternatively, the core shell particle 100 has one layer of therapeutic agent positioned between two biodegradable layers 102a,b.

In at least one embodiment, the core shell particle 100 comprises at least two layers of therapeutic agent 30 and at least two biodegradable layers 102. One possible configuration of this embodiment is shown in FIG. 45D. The core shell particle 100 in FIG. 45D has a first section/layer of therapeutic agent 30a, a first biodegradable layer 102a around the first section of therapeutic agent 30a, a second therapeutic agent 30b layer around the first biodegradable layer 102a and a second biodegradable layer 102b around the second therapeutic agent 30b layer.

The core shell particles 100 can be deposited onto a member 14 by dip-coating, pressure filling, particle printing, laser transfer from foils, a rolling process or by another other means known in the art. In at least one embodiment, core shell particles 100 are removed from specific areas of the stent 10. This allows the elution of therapeutic agent 30 from selected portions of the stent 10 while other portions of the stent 10 do not elute therapeutic agent 30.

In at least one embodiment, a chemical is used to flush the holes 44 of a coating retainer 22 before the therapeutic agent 30 is deposited into the coating retainer 22. Examples of chemicals that can be used to flush the holes 44 include, but are not limited to, toluene, tetrahydrofuran (THF), dimethylformamide (DMF), and water. Any therapeutic agent 30 deposited onto the surface of the member 14 that comes into contact with the chemical will swell into the hole 44. In at least one embodiment, a chemical is used to flush the holes 44 of a coating retainer 22 before the therapeutic agent 30 is deposited into the coating retainer 22 so that the therapeutic agent 30 is retained in the holes 44 by the chemical. Examples of chemicals that will retain the therapeutic agent 30 in the holes, include, but are not limited to, cyclodextrins, amphiphilic structures, reactive molecules. Amphiphilic structures include, but are not limited to, surfactant micelles and lipid micelles. Reactive molecules include, but are not limited to, molecules that covalently react with the therapeutic agent 30, e.g. cross-linking chemicals such as hydrazone linker or disulfide linker, antibodies to a specific antigen, antigens to a specific antibody, and a chemical which reacts with part of the therapeutic agent 30 in a precursor form so that the chemical is activated, e.g. zymogen or a proenzyme that is in an inactive form until it undergoes a biochemical change to an active enzyme.

These methods of increasing adhesion can be used with coating retainer 22 embodiments illustrated, for example in FIGS. 9-11. Note that if the holes 44 are made in the member 14 before polishing of the stent, the holes 44 will have tapered ostiums. In the case of holes 44 extending through the body of the member 14 from one surface to the opposite surface, the hole 44 will have an hour-glass shape. In this embodiment, the therapeutic agent 30 can swell down into the hole 44 beyond the “waist” of the hour-glass shaped hole 44.

In at least one embodiment, the therapeutic agent 30 is applied to the stent 10 as a polymer film. This method can be used with roughened surfaces, such as is seen, for example, with the metal oxide 52 embodiment of FIG. 21 or with the grit blasting technique described above. Alternatively, the method can be used, for example, with channels 40, with wells 42, or with protrusions 48.

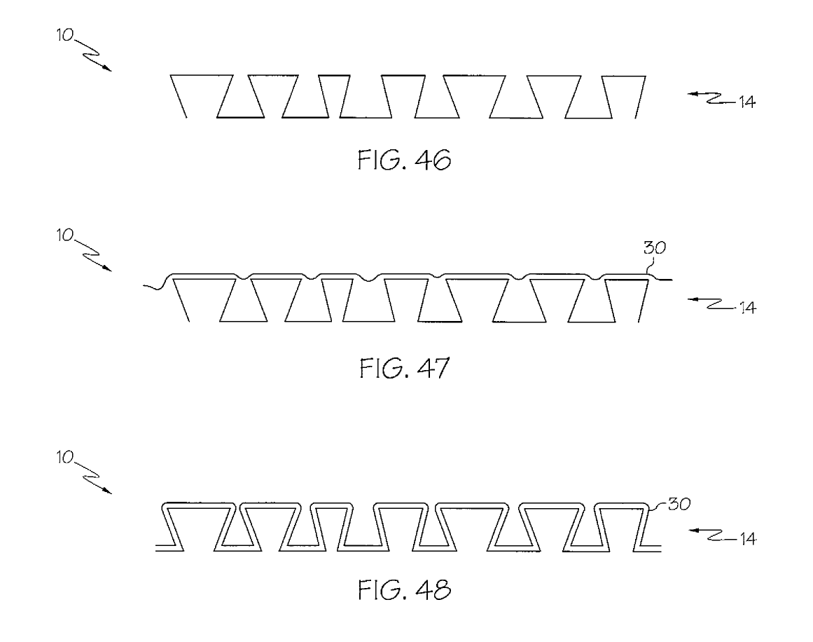

FIGS. 46-48 show one method of applying a therapeutic agent 30 to a polymer film to the stent 10. FIG. 46 is a non-limiting example of the irregular surface of the member 14 to which this method is directed. The first step in this method is to deposit a polymer film of therapeutic agent 30, hereinafter polymer film 30, onto the surface of the stent 10. Usually the polymer film 30 is applied to the stent 10 as a solution which is mostly solvent. As the solvent evaporates, there is shrinkage of the polymer film 30 which causes the polymer film 30 to pull up and away from the surface of the stent 10. When the polymer film 30 pulls away from the surface of the stent 10, maximum adhesion of the polymer film 30 to the stent 10 has not been achieved. Maximum adhesion has not been achieved because there are pockets of air that are trapped in the “valleys” beneath the polymer film 30. These trapped pockets of air act as fluid reservoirs, thereby causing undercutting and accelerating delamination of the polymer film 30. Delamination occurs because, when fluid penetrates at the interface between the polymer film 30 and the surface of the stent 10, the forces of adhesion due to all non-covalent interactions that enhance adhesion, including acid-based interaction, hydrogen bonding, etc. are displaced by the hydration interactions, thereby lessening the adhesive force between substrate and coating, engaging the polymer film 30 to the surface of the stent 10.

In contrast, the polymer film 30 used in this method is a 100% solids film which means that there is no solvent. The polymer film 30 can be applied to the stent 10 by any conventional means, for example, but not limited to, spraying, roll coating, thermal processing, or ink jet printing. FIG. 47 illustrates how the polymer film 30 initially coats the stent 10. After the polymer film 30 is deposited, the stent 10 is placed into a vacuum oven. In the vacuum oven, the temperature of the polymer film 30 is raised above its softening point and the pressure is cyclically reduced and raised back to atmospheric. As a non-limiting example, for a SIBS coating (polystyrene-b-polyisobutylene-b-polystyrene block copolymer), which has a softening point near 120° C., the oven is heated to 125° C. This causes the polymer film 30 to flow down into the “valleys” because the gas that was trapped in the “valleys” underneath the polymer film 30 is pulled out of the “valleys” by the vacuum. FIG. 48 illustrates how the polymer film 30 is in close contact with surface area of the roughened stent 10. This close contact minimizes the penetration of water/fluids which thereby minimizes the possibility of delamination of the polymer film 30 from the stent 10. Thus, the topography of the surface of the stent 10 maximizes the adhesive strength of the polymer film 30 to the stent 10 through mechanical interlocking.

Another method by which a therapeutic agent 30 can be engaged to a stent 10 with a coating retainer 22 is to apply the therapeutic agent 30, such as a drug coating, above glass transition temperature (T_{G}) in the case of an amorphous polymer or above the melt temperature (Tm) in the case of a semicrystalline polymer. At this temperature, the drug coating readily flows into coating retainers 22 positioned within the body of the member 14, for example, but not limited to channels 40. After the drug coating has been applied to the stent 10, the drug coating is allowed to cool such that the therapeutic agent can no longer flow. In some embodiments, when the drug coating cools, it volumetrically expands thereby engaging the therapeutic agent 30 to the stent 10.

Another method by which a therapeutic agent 30 can be engaged to a stent 10 with a coating retainer 22 is to use a two part curing system. In this method, the therapeutic agent 30 is applied to the stent 10 in a low viscosity state. When the therapeutic agent 30 cures into a solid film, it adheres to the stent 10. In some embodiments, the therapeutic agent 30 adheres by mechanical interlocking.

Another method to engage a therapeutic agent 30 to a stent 10 utilizes heat. In this method, the stent 10 is heated so that coating retainers 22, such as channels 40 for example, become enlarged. Note that the temperature range used depends on the thermal expansion coefficient of the metal and the polymer of the therapeutic agent 30. In at least one embodiment, the stent material has a higher thermal expansion coefficient than the excipient polymer, i.e. the inert substance used as a vehicle for a drug, containing the therapeutic agent 30. While the coating retainers 22 are enlarged, a therapeutic agent 30, for example a drug coating, is applied to the stent 10. After the therapeutic agent 30 is applied, the stent 10 is allowed to cool which causes the coating retainers 22 to contract, thereby engaging (or trapping depending upon the structure of the coating retainer 22) the therapeutic agent 30 to the stent 10.

The following numbered statements characterize embodiments described above:

1. A stent, the stent comprising a plurality of members, at least one of the plurality of members comprising a first mechanism to reduce delamination of a substance and a second mechanism to reduce delamination of a substance, the first mechanism being different than the second mechanism, wherein the first mechanism to reduce delamination of a substance is selected from at least one member of the group consisting of holes, protrusions, stainless steel shields, clamps, pins, porous material and any combination thereof, and the second mechanism to reduce delamination of a substance is selected from at least one member of the group consisting of holes, protrusions, stainless steel shields, clamps, pins, porous material and any combination thereof.

2. The stent of statement 1, the substance selected from at least one member of the group consisting of non-genetic therapeutic agents, genetic therapeutic agents, cellular material, polymer agent and any combination thereof

3. The stent of statement 1, the at least one of the plurality of members being a first member, the first member having a body, the body having at least one surface, a length, a width and a thickness;
the first mechanism being at least one hole, the at least one hole being a channel, the body the first member defining the at least one channel, the at least one channel comprising a first channel in the at least one surface of the first member, the first channel comprising an opening in the at least one surface of the body of the first member, a first side, a second side, a third side, a fourth side and a bottom surface, the distance from the at least one surface of the body of the first member to the bottom surface of the first channel determining a first depth, the first depth less than the thickness of the body of the first member, the distance from the first side to the third side determining a first width, the first width less than the width of the body of the first member, the distance from the second side to the fourth side determining a first length, the first length less than the length of the body of the first member;
the second mechanism being at least one hole, the body of the first member further defining the at least one hole, the at least one hole comprising a first hole, the first hole at a first oblique angle to the at least one surface of the first member, the first hole having a first depth, the first depth at most equal to the thickness of the body of the first member, the at least one hole further comprising a second hole, the second hole at a second oblique angle to the at least one surface of the first member, the first hole and the second hole forming a passageway, the passageway having a configuration selected from at least one member of the group consisting of V-shaped, U-shaped, Y-shaped, y-shaped, X-shaped, L-shaped, T-shaped, irregular shaped and any combination thereof.

4. The stent of statement 3, the opening of the first channel having a width, the bottom surface of the first channel having a width, the width of the opening less than the width of the bottom surface.

5. The stent of statement 3, the at least one channel further comprising a second channel, the second channel comprising an opening in the bottom surface of the first channel, a first side, a second side, a third side, a fourth side and a bottom surface, the distance from the bottom surface of the first channel to the bottom surface of the second channel determining a second depth, the first and second depth forming a total depth, the total depth less than the thickness of the body of the first member, the distance from the first side to the third side of the second channel determining a second width, the second width less than the first width, the distance from the second side to the fourth side of the second channel determining a second length, the second length less than the first length.

6. The stent of statement 1, the at least one of the plurality of members being a first member, the first member having a body, the body having at least one surface, a length, a width and a thickness;
the first mechanism being at least one hole, the at least one hole being a channel, the body of the first member defining the at least one channel, the at least one channel comprising a first channel in the at least one surface of the first member, the first channel comprising an opening in the at least one surface of the body of the first member, a first side, a second side, a third side, a fourth side and a bottom surface, the distance from the at least one surface of the body of the first member to the bottom surface of the first channel determining a first depth, the first depth less than the thickness of the body of the first member, the distance from the first side to the third side determining a first width, the first width less than the width of the body of the first member, the distance from the second side to the fourth side determining a first length, the first length less than the length of the body of the first member;
the second mechanism being at least one protrusion, the at least one surface of the first member having the at least one protrusion, the at least one protrusion comprising a body, the body of the at least one protrusion having a configuration selected from at least one member of the group consisting of substantially round, substantially oval, substantially square, substantially rectangular, substantially triangular, substantially octagonal, substantially polygon, a plurality of peaks and valleys, and any combination thereof.

7. The stent of statement 6, the at least one protrusion forming at least one channel, the at least one surface of the first member having a length and comprising a first region, a second region, and a third region, the at least one protrusion comprising a first protrusion and a second protrusion, the first and second protrusions each having a length, the length being less than the length of the at least one surface of the first member, the first protrusion engaged to the first edge region, the second protrusion engaged to the second edge region, the third region of the at least one surface being between the first wall and the second wall, the third region forming the bottom surface of the at least one channel, the first and second protrusions forming opposite side of the at least one channel.

8. The stent of statement 6, the body of the at least one protrusion having a cap, the cap having the same configuration as the body of the at least one protrusion, the body of the at least one protrusion having a top surface, the top surface being horizontal to the at least one surface of the first member.

9. The stent of statement 6, the body of the at least one protrusion having a cap, the cap having a configuration different than the body of the at least one protrusion, the at least one protrusion having a cross-section selected from at least one member of the group consisting of barb-like, mushroom-shaped, arrow-shaped, T-shaped, P-shaped, and any combination thereof.

10. The stent of statement 6, the plurality of peaks and valleys of the at least one protrusion being formed by at least one metal oxide, the at least one metal oxide selected from at least one member of the group consisting of aluminum oxide, magnesium oxide, iron oxide, iridium oxide (Irox), iridium-iridium oxide (Ir—Irox), titanium oxide, titanium-iridium-iridium oxide (Ti—Ir—Irox), titanium-nitrogen oxide (TiNOx), titanium-titanium nitrogen oxide (Ti—TiNOx), tantalum oxide, tungsten oxide, niobium oxide, and any combination thereof.

11. The stent of statement 1, the at least one of the plurality of members being a first member, the first member having a body, the body having at least one surface, a length, a width and a thickness;
the first mechanism being at least one hole, the at least one hole being a channel, the body of the first member defining the at least one channel, the at least one channel comprising a first channel in the at least one surface of the first member, the first channel comprising an opening in the at least one surface of the body of the first member, a first side, a second side, a third side, a fourth side and a bottom surface, the distance from the at least one surface of the body of the first member to the bottom surface of the first channel determining a first depth, the first depth less than the thickness of the body of the first member, the distance from the first side to the third side determining a first width, the first width less than the width of the body of the first member, the distance from the second side to the fourth side determining a first length, the first length less than the length of the body of the first member;
the second mechanism being at least one shield, the at least one surface of the first member having the at least one shield, the at least one shield having an exterior surface and comprising a transverse portion and an elongated portion, the elongated portion parallel to the body of the first member, the transverse portion at an oblique angle to the body of the first member.

12. The stent of statement 11, the at least one shield further comprising a cap, the cap engaged to the transverse portion, at least a portion of the cap extending over a portion of the at least one surface of the first member.

13. The stent of statement 1, the at least one of the plurality of members comprising a first member, the first member having a body, the body having at least one surface, a length, a width and a thickness;
the first mechanism being at least one hole, the at least one hole being a channel, the body of the first member defining the at least one channel, the at least one channel comprising a first channel in the at least one surface, the first channel comprising an opening in the at least one surface of the first member, a first side, a second side, a third side, a fourth side and a bottom surface, the distance from the at least one surface of the body to the bottom surface of the first channel determining a first depth, the first depth less than the thickness of the body of the first member, the distance from the first side to the third side determining a first width, the first width less than the width of the body of the first member, the distance from the second side to the fourth side determining a first length, the first length less than the length of the body of the first member;
the second mechanism being at least one clamp, the body of the first member having the at least one clamp engaged thereto, the at least one clamp comprising a first clamp, the first clamp having a body comprising a width, a first region and a second region, the width of the body of the first clamp greater than the width of the body of the first member, the first elanip further having a first arm and a second arm, the first arm engaged to the body of the first clamp at the first region, the second arm engaged to the body of the first clamp at the second region, the first and second arms extending at an oblique angle to the body of the first clamp, the first arm, the second arm and the body of the first clamp engaging the first clamp to the body of the first member.

14. The stent of statement 1, the at least one of the plurality of members being a first member, the first member having a body, the body having at least one surface, a length, a width and a thickness;
the first mechanism being at least one hole, the at least one hole being a channel, the body of the first member defining the at least one channel, the first channel comprising an opening in the at least one surface of the body of the first member, a first side, a second side, a third side, a fourth side and a bottom surface, the distance from the at least one surface of the body of the first member to the bottom surface of the first channel determining a first depth, the first depth less than the thickness of the body of the first member, the distance from the first side to the third side determining a first width, the first width less than the width of the body of the first member, the distance from the second side to the fourth side determining a first length, the first length less than the length of the body of the first member;
the second mechanism being at least one pin, the at least one pin comprising a first pin, the first pin comprising a shaft and a head, the first member defining at least one hole, the at least one hole comprising a first hole, the first hole having a complementary shape to the shaft of the first pin so that the shaft can be inserted into the first hole.

15. The stent of statement 14, the first pin having a configuration, the configuration selected from at least one member of the group consisting of push-pin, thumb-tack and any combination thereof.

16. The stent of statement 1, the at least one of the plurality of members being a first member, the first member having a body, the body having at least one surface, a length, a width and a thickness;
the first mechanism being at least one hole, the at least one hole being a channel, the body of the first member defining the at least one channel, the at least one channel comprising a first channel in the at least one surface of the first member, the first channel comprising an opening in the at least one surface of the first member, a first side, a second side, a third side, a fourth side and a bottom surface, the distance from the first surface of the body of the first member to the bottom surface of the first channel determining a first depth, the first depth less than the thickness of the body of the first member, the distance from the first side to the third side determining a first width, the first width less than the width of the body of the first member, the distance from the second side to the fourth side determining a first length, the first length less than the length of the body of the first member;
the second mechanism being a porous material, at least a portion of the body of the first member comprising the porous material.

17. The stent of statement 1, the at least one of the plurality of members being a first member, the first member having a body, the body having at least one surface, a length, a width and a thickness;
the first mechanism being at least one hole, the body of the first member defining the at least one hole, the at least one hole comprising a first hole, the first hole at a first oblique angle to the at least one surface of the first member, the first hole having a first depth, the first depth at most equal to the thickness of the body of the first member;
the second mechanism being at least one protrusion, the at least one surface of the first member having the at least one protrusion, the at least one protrusion comprising a body, the body of the at least one protrusion having a configuration selected from at least one member of the group consisting of substantially round, substantially oval, substantially square, substantially rectangular, substantially triangular, substantially octagonal, substantially polygon, a plurality of peaks and valleys, and any combination thereof.

18. The stent of statement 17, the at least one protrusion forming at least one channel, the at least one surface of the first member having a length and comprising a first region, a second region, and a third region, the at least one protrusion comprising a first protrusion and a second protrusion, the first and second protrusions each having a length, the length being less than the length of the at least one surface of the first member, the first protrusion engaged to the first edge region, the second protrusion engaged to the second edge region, the third region of the first surface being between the first wall and the second wall, the third region forming the bottom surface of the at least one channel, the first and second protrusions forming opposite side of the at least one channel.

19. The stent of statement 17, the body of the at least one protrusion having a cap, the cap having the same configuration as the body of the at least one protrusion, the body of the at least one protrusion having a top surface, the top surface being horizontal to the at least one surface of the first member.

20. The stent of statement 17, the body of the at least one protrusion having a cap, the cap having a configuration different than the body of the at least one protrusion, the at least one protrusion having a cross-section selected from at least one member of the group consisting of barb-like, mushroom-shaped, arrow-shaped, T-shaped, P-shaped, and any combination thereof.

21. The stent of statement 17, the plurality of peaks and valleys of the at least one protrusion being formed by at least one metal oxide, the at least one metal oxide selected from at least one member of the group consisting of aluminum oxide, magnesium oxide, iron oxide, iridium oxide (Irox), iridium-iridium oxide (Ir—Irox), titanium oxide, titanium-iridium-iridium oxide (Ti—Ir—Irox), titanium-nitrogen oxide (TiNOx), titanium-titanium nitrogen oxide (Ti—TiNOx), tantalum oxide, tungsten oxide, niobium oxide, and any combination thereof.

22. The stent of statement 17, the at least one hole further comprising a second hole, the second hole at a second oblique angle to the at least one surface of the first member, the first hole and the second hole forming a passageway, the passageway having a configuration selected from at least one member of the group consisting of V-shaped, U-shaped, Y-shaped, y-shaped, X-shaped, L-shaped, T-shaped, irregular shaped and any combination thereof.

23. The stent of statement 17, the at least one surface of the first member comprising a first surface and a second surface, the at least one hole further comprising a second hole, the first hole at a first oblique angle to the first surface of the first member, the second hole at a second oblique angle to the second surface of the first member, the second hole having a second depth, the second depth less than the thickness of the body of the first surface.

24. The stent of statement 23, the first surface opposite from the second surface, the first member further comprising a third surface, the third surface between the first and second surfaces.

25. The stent of statement 23, the first depth greater than the second depth.

26. The stent of statement 1, the at least one of the plurality of members being a first member, the first member having a body, the body having at least one surface, a length, a width and a thickness;
the first mechanism being at least one hole, the body of the first member defining the at least one hole, the at least one hole comprising a first hole, the first hole at a first oblique angle to the at least one surface, the first hole having a first depth, the first depth at most equal to the thickness of the body of the first member;
the second mechanism being at least one shield, the at least one surface having the at least one shield, the at least one shield having an exterior surface and comprising a transverse portion and an elongated portion, the elongated portion parallel to the body, the transverse portion at an oblique angle to the body of the first member.

27. The stent of statement 26, the at least one shield further comprising a cap, the cap engaged to the transverse portion, at least a portion of the cap extending over a portion of the at least one surface of the first member.

28. The stent of statement 1, the at least one of the plurality of members being a first member, the first member having a body, the body having at least one surface, a length, a width and a thickness;
the first mechanism being at least one hole, the body of the first member defining the at least one hole, the at least one hole comprising a first hole, the first hole at a first oblique angle to the at least one surface, the first hole having a first depth, the first depth at most equal to the thickness of the body of the first member;
the second mechanism being at least one clamp, the body of the first member having the at least one clamp engaged thereto, the at least one clamp having a body comprising a width, a first region and a second region, the width of the body of the at least one clamp greater than the width of the body of the first member, the at least one clamp further having a first arm and a second arm, the first arm engaged to the body of the at least one clamp at the first region, the second arm engaged to the body of the at least one clamp at the second region, the first and second arms extending at an oblique angle to the body of the clamp, the first arm, the second arm and the body of the at least one clamp engaging the at least one clamp to the body of the first member.

29. The stent of statement 1, the at least one of the plurality of members being a first member, the first member having a body, the body having at least one surface, a length, a width and a thickness;
the first mechanism being at least one hole, the body of the first member defining the at least one hole, the at least one hole comprising a first hole, the first hole at a first oblique angle to the at least one surface, the first hole having a first depth, the first depth at most equal to the thickness of the body of the first member;
the second mechanism being at least one pin, the at least one pin comprising a shaft and a head, the body of the first member defining at least one hole, the at least one hole having a complementary shape to the shaft of the at least one pin so that the shaft can be inserted into the at least one hole.

30. The stent of statement 29, the at least one pin having a configuration, the configuration selected from at least one member of the group consisting of push-pin, thumb-tack and any combination thereof.

31. The stent of statement 1, the at least one of the plurality of members being a first member, the first member having a body, the body having at least one surface, a length, a width and a thickness;
the first mechanism being at least one hole, the body of the first member defining the at least one hole, the at least one hole comprising a first hole, the first hole at a first oblique angle to the at least one surface, the first hole having a first depth, the first depth at most equal to the thickness of the body of the first member;
the second mechanism being a porous material, the first member comprising a first portion, the first portion of the body of the first member comprising the porous material.

32. The stent of statement 31, wherein the porous material is sintered metal.

33. The stent of statement 31, the first portion of the body of the first member further comprising a non-porous material, the first portion of the body of the first member having a first layer, a second layer and a third layer, the first layer having a top surface, the first and second layers being non-porous material, a second layer being porous material, the second layer positioned between the first layer and the third layer, the first layer of non-porous material defining at least one hole, the at least one hole extending from the second layer to the top surface of the first layer.

34. The stent of statement 33, the first portion of the body of the first member further comprising a coating, the coating surrounding the first, second and third layers of the first portion of the body of the first member.

35. The stent of statement 34, the coating being biodegradable.

36. The stent of statement 34, the coating defining at least one hole, the at least one hole in the coating contiguous with the at least one hole in the first layer.

37. The stent of statement 35, the first portion of the body of the first member further comprising a non-porous material and a coating, the porous material surrounding the non-porous material, the coating surrounding the non-porous material.

38. The stent of statement 37, the coating having a top surface, the coating defining at least one hole, the at least one hole extending from the porous material to the top surface of the coating.

39. The coating of statement 37, the coating being biodegradable.

40. The stent of statement 1, the at least one of the plurality of members being a first member, the first member having a body, the body having at least one surface, a length, a width and a thickness;
the first mechanism being at least one protrusion, the at least one surface of the first member having the at least one protrusion, the at least one protrusion comprising a body, the body of the at least one protrusion having a configuration selected from at least one member of the group consisting of substantially round, substantially oval, substantially square, substantially rectangular, substantially triangular, substantially octagonal, substantially polygon, a plurality of peaks and valleys, and any combination thereof;
the second mechanism being at least one shield, the at least one surface of the first member having the at least one shield, the at least one shield having an exterior surface and comprising a transverse portion and an elongated portion, the elongated portion parallel to the body of the first member, the transverse portion at an oblique angle to the body of the first member.

41. The stent of statement 40, the at least one shield further comprising a cap, the cap engaged to the transverse portion, at least a portion of the cap extending over a portion of the at least one surface of the first member.

42. The stent of statement 1, the at least one of the plurality of members being a first member, the first member having a body, the body having at least one surface, a length, a width and a thickness;
the first mechanism being at least one protrusion, the at least one surface of the first member having the at least one protrusion, the at least one protrusion comprising a body, the body of the at least one protrusion having a configuration selected from at least one member of the group consisting of substantially round, substantially oval, substantially square, substantially rectangular, substantially triangular, substantially octagonal, substantially polygon, a plurality of peaks and valleys, and any combination thereof;
the second mechanism being at least one clamp, the body of the first member having the at least one clamp engaged thereto, the at least one clamp having a body comprising a width, a first region and a second region, the width of the body of the at least one clamp greater than the width of the body of the first member, the at least one clamp further having a first arm and a second arm, the first arm engaged to the body of the clamp at the first region, the second arm engaged to the body of the clamp at the second region, the first and second arms extending at an oblique angle to the body of the clamp, the first arm, the second arm and the body of the at least one clamp engaging the at least one clamp to the body of the first member.

43. The stunt of statement 1, the at least one of the plurality of members being a first member, the first member having a body, the body having at least one surface, a length, a width and a thickness;
the first mechanism being at least one protrusion, the at least one surface of the first member having the at least one protrusion, the at least one protrusion comprising a body, the body of the at least one protrusion having a configuration selected from at least one member of the group consisting of substantially round, substantially oval, substantially square, substantially rectangular, substantially triangular, substantially octagonal, substantially polygon, a plurality of peaks and valleys, and any combination thereof;
the second mechanism being at least one pin, the at least one pin comprising a shaft and a head, the body of the first member defining at least one hole, the at least one hole having a complementary shape to the shaft of the at least one pin so that the shaft can be inserted into the at least one hole.

44. The stent of statement 43, the at least one pin having a configuration, the configuration selected from at least one member of the group consisting of push-pin, thumb-tack and any combination thereof.

45. The stent of statement 1, the at least one of the plurality of members being a first member, the first member having a body, the body having at least one surface, a length, a width and a thickness;
the first mechanism being at least one protrusion, the at least one surface of the first member having the at least one protrusion, the at least one protrusion comprising a body, the body of the at least one protrusion having a configuration selected from at least one member of the group consisting of substantially round, substantially oval, substantially square, substantially rectangular, substantially triangular, substantially octagonal, substantially polygon, a plurality of peaks and valleys, and any combination thereof;
the second mechanism being a porous material, the first member comprising a first portion, the first portion of the body of the first member comprising the porous material.

46. The stent of statement 45, wherein the porous material is sintered metal.

47. The stent of statement 45, the first portion of the body of the first member further comprising a non-porous material, the first portion of the body of the first member having a first layer, a second layer and a third layer, the first layer having a top surface, the first and second layers being non-porous material, a second layer being porous material, the second layer positioned between the first layer and the third layer, the first layer of non-porous material defining at least one hole, the at least one hole extending from the second layer to the top surface of the first layer.

48. The stent of statement 47, the first portion of the body of the first member further comprising a coating, the coating surrounding the first, second and third layers of the first portion of the body of the first member.

49. The stent of statement 48, the coating being biodegradable.

50. The stent of statement 48, the coating defining at least one hole, the at least one hole in the coating contiguous with the at least one hole in the first layer.

51. The stent of statement 45, the first portion of the body of the first member further comprising a non-porous material and a coating, the porous material surrounding the non-porous material, the coating surrounding the non-porous material.

52. The stent of statement 51, the coating having a top surface, the coating defining at least one hole, the at least one hole extending from the porous material to the top surface of the coating.

53. The coating of statement 51, the coating being biodegradable,

54. The stent of statement 45, the at least one protrusion forming at least one channel, the first member having at least one surface, the at least one surface of the first member having a length and comprising a first region, a second region, and a third region, the at least one protrusion comprising a first protrusion and a second protrusion, the first and second protrusions each having a length, the length being less than the length of the at least one surface of the first member, the first protrusion engaged to the first edge region, the second protrusion engaged to the second edge region, the third region of the at least one surface of the first member being between the first wall and the second wall, the third region forming the bottom surface of the at least one channel, the first and second protrusions forming opposite side of the at least one channel.

55. The stent of statement 45, the body of the at least one protrusion having a cap, the cap having the same configuration as the body of the at least one protrusion, the body of the at least one protrusion having a top surface, the top surface of the at least one protrusion being horizontal to the at least one surface of the first member.

56. The stent of statement 45, the body of the at least one protrusion having a cap, the cap having a configuration different than the body of the at feast one protrusion, the at least one protrusion having a cross-section selected from at least one member of the group consisting of barb-like, mushroom-shaped, arrow-shaped, T-shaped, P-shaped, and any combination thereof.

57. The stent of statement 45, the plurality of peaks and valleys of the at least one protrusion, being formed of at least one metal oxide, the at least one metal oxide selected from at least one member of the group consisting of aluminum oxide, magnesium oxide, iron oxide, iridium oxide (Irox), iridium-iridium oxide (Ir—Irox), titanium oxide, titanium-iridium-iridium oxide (Ti—Ir—Irox), titanium-nitrogen oxide (TiNOx), titanium-titanium nitrogen oxide (Ti—TiNOx), tantalum oxide, tungsten oxide, niobium oxide, and any combination thereof.

58. The stent of statement 1, the at least one of the plurality of members being a first member, the first member having a body, the body having at least one surface, a length, a width and a thickness;
the first mechanism being at least one shield, the at least one surface of the first member having the at least one shield, the at least one shield having an exterior surface and comprising a transverse portion and an elongated portion, the elongated portion parallel to the body of the first member, the transverse portion at an oblique angle to the body of the first member;
the second mechanism being at least one clamp, the body of the first member having the at least one clamp engaged thereto, the at least one clamp having a body comprising a width, a first region and a second region, the width of the body of the at least one clamp greater than the width of the body of the first member, the at least one clamp further having a first arm and a second arm, the first arm engaged to the body of the at least one clamp at the first region, the second arm engaged to the body of the at least one clamp at the second region, the first and second arms extending at an oblique angle to the body of the at least one clamp, the first arm, the second arm and the body of the at least one clamp engaging the at least one clamp to the body of the first member.

59. The stent of statement 1, the at least one of the plurality of members being a first member, the first member having a body, the body having at least one surface, a length, a width and a thickness;
the first mechanism being at least one shield, the at least one surface of the first member having the at least one shield, the at least one shield having an exterior surface and comprising a transverse portion and an elongated portion, the elongated portion parallel to the body of the first member, the transverse portion at an oblique angle to the body of the first member;
the second mechanism being at least one pin, the at least one pin comprising a shaft and a head, the body of the first member defining at least one hole, the at least one hole having a complementary shape to the shaft of the at least one pin so that the shaft can be inserted into the at least one hole.

60. The stent of statement 59, the at least one pin having a configuration, the configuration selected from at least one member of the group consisting of push-pin, thumb-tack and any combination thereof.

61. The stent of statement 1, the at least one of the plurality of members being a first member, the first member having a body, the body having at least one surface, a length, a width and a thickness;
the first mechanism being at least one shield, the at least one surface of the first member having the at least one shield, the at least one shield having an exterior surface and comprising a transverse portion and an elongated portion, the elongated portion parallel to the body of the first member, the transverse portion at an oblique angle to the body of the first member;
the second mechanism being a porous material, the first member comprising a first portion, the first portion of the body of the first member comprising the porous material.

62. The stent of statement 61, wherein the porous material is sintered metal.

63. The stent of statement 61, the first portion of the body of the first member further comprising a non-porous material, the first portion of the body of the first member having a first layer, a second layer and a third layer, the first layer having a top surface, the first and second layers being non-porous material, a second layer being porous material, the second layer positioned between the first layer and the third layer, the first layer of non-porous material defining at least one hole, the at least one hole extending from the second layer to the top surface of the first layer.

64. The stent of statement 63, the first portion of the body of the first member further comprising a coating, the coating surrounding the first, second and third layers of the first portion of the body of the first member.

65. The stent of statement 64, the coating being biodegradable.

66. The stent of statement 64, the coating defining at least one hole, the at least one hole in the coating contiguous with the at least one hole in the first layer.

67. The stent of statement 61, the first portion of the body of the first member further comprising a non-porous material and a coating, the porous material surrounding the non-porous material, the coating surrounding the non-porous material.

68. The stent of statement 67, the coating having a top surface, the coating defining at least one hole, the at least one hole extending from the porous material to the top surface of the coating.

69. The coating of statement 67, the coating being biodegradable.

70. The stent of statement 61, the at least one shield further comprising a cap, the cap engaged to the transverse portion, at least a portion of the cap extending over a portion of the at least one surface of the first member.

71. The stent of statement 1, the at least one of the plurality of members being a first member, the first member having a body, the body having at least one surface, a length, a width and a thickness;
the first mechanism being at least one clamp, the body of the first member having the at least one clamp engaged thereto, the at least one clamp having a body comprising a width, a first region and a second region, the width of the body of the at least one clamp greater than the width of the body of the first member, the at least one clamp further having a first arm and a second arm, the first arm engaged to the body of the at least one clamp at the first region, the second arm engaged to the body of the at least one clamp at the second region, the first and second arms extending at an oblique angle to the body of the at least one clamp, the first arm, the second arm and the body of the at least one clamp engaging the at least one clamp to the body of the first member;
the second mechanism being at least one pin, the at least one pin comprising a shaft and a head, the body of the first member defining at least one hole, the at least one hole having a complementary shape to the shaft of the at least one pin so that the shaft can be inserted into the at least one hole.

72. The stent of statement 71, the at least one pin having a configuration, the configuration selected from at least one member of the group consisting of push-pin, thumb-tack and any combination thereof.

73. The stent of statement 1, the at least one of the plurality of members being a first member, the first member having a body, the body having at least one surface, a length, a width and a thickness;
the first mechanism being at least one clamp, the body of the first member having the at least one clamp engaged thereto, the at least one clamp having a body comprising a width, a first region and a second region, the width of the body of the at least one clamp greater than the width of the body, the at least one clamp further having a first arm and a second arm, the first arm engaged to the body of the at least one clamp at the first region, the second arm engaged to the body of the at least one clamp at the second region, the first and second arms extending at an oblique angle to the body of the at least one clamp, the first arm, the second arm and the body of the at least one clamp engaging the at least one clamp to the body of the first member;
the second mechanism being a porous material, the first member comprising a first portion, the first portion of the body of the first member comprising the porous material.

74. The stent of statement 73, wherein the porous material is sintered metal.

75. The stent of statement 73, the first portion of the body of the first member further comprising a non-porous material, the first portion of the body of the first member having a first layer, a second layer and a third layer, the first layer having a top surface, the first and second layers being non-porous material, a second layer being porous material, the second layer positioned between the first layer and the third layer, the first layer of non-porous material defining at least one hole, the at least one hole extending from the second layer to the top surface of the first layer.

76. The stent of statement 75, the first portion of the body of the first member further comprising a coating, the coating surrounding the first, second and third layers of the first portion of the body of the first member.

77. The stent of statement 76, the coating being biodegradable.

78. The stent of statement 76, the coating defining at least one hole, the at least one hole in the coating contiguous with the at least one hole in the first layer.

79. The stent of statement 73, the first portion of the body of the first member further comprising a non-porous material and a coating, the porous material surrounding the non-porous material, the coating surrounding the non-porous material.

80. The stent of statement 79, the coating having a top surface, the coating defining at least one hole, the at least one hole extending from the porous material to the top surface of the coating.

81. The coating of statement 79, the coating being biodegradable.

82. The stent of statement 1, the at least one of the plurality of members being a first member, the first member having a body, the body having at least one surface, a length, a width and a thickness;
the first mechanism being at least one pin, the at least one pin comprising a shaft and a head, the body of the first member defining at least one hole, the at least one hole having a complementary shape to the shaft of the at least one pin so that the shaft can be inserted into the at least one hole;
the second mechanism being a porous material, the first member comprising a first portion, the first portion of the body of the first member comprising the porous material.

83. The stent of statement 82, wherein the porous material is sintered metal.

84. The stent of statement 82, the first portion of the body of the first member further comprising a non-porous material, the first portion of the body of the first member having a first layer, a second layer and a third layer, the first layer having a top surface, the first and second layers being non-porous material, a second layer being porous material, the second layer positioned between the first layer and the third layer, the first layer of non-porous material defining at least one hole, the at least one hole extending from the second layer to the top surface of the first layer.

85. The stent of statement 84, the first portion of the body of the first member further comprising a coating, the coating surrounding the first, second and third layers of the first portion of the body of the first member.

86. The stent of statement 85, the coating being biodegradable.

87. The stent of statement 85, the coating defining at least one hole, the at least one hole in the coating contiguous with the at least one hole in the first layer.

88. The stent of statement 82, the first portion of the body of the first member further comprising a non-porous material and a coating, the porous material surrounding the non-porous material, the coating surrounding the non-porous material.

89. The stent of statement 88, the coating having a top surface, the coating defining at least one hole, the at least one hole extending from the porous material to the top surface of the coating.

90. The coating of statement 88, the coating being biodegradable.

91. The stent of statement 82, the at least one pin having a configuration, the configuration selected from at least one member of the group consisting of push-pin, thumb-tack and any combination thereof.

The following numbered statements characterize embodiments described above:

1. A stent, the stent comprising
a plurality of members, each of the plurality of members having a body, the body having an volume, the body defining a first channel and a second channel therein, the first channel having a first volume, the second channel having a second volume, the first volume different than the second volume and the cumulative volumes of the first volume and the second volume less than the volume of the body;
first therapeutic agent, the first therapeutic agent deposited within the first channel; and
a second therapeutic agent, the second therapeutic agent deposited within the second channel.

2. The stent of statement 1, wherein
the body of each of the plurality of members having at least one surface, each body having a length, width and thickness, at least one of the plurality of members defining the first channel in the at least one surface of the body and the second channel in the at least one surface of the body;
the first channel comprising an opening in the at least one surface of the body, a first side, a second side, a third side, and a fourth side, and a bottom surface, the distance from the at least one surface of the body to the bottom surface of the first channel determining a first depth, the first depth less than the thickness of the body, the distance from the first side to the third side determining a first width, the first width less than the width of the body, the distance from the second side to the fourth side determining a first length, the first length less than the length of the body but at least one quarter of the length of the body, the first volume determined by the first depth, first length and first width; and
the second channel comprising an opening in the at least one surface of the body, a first side, a second side, a third side, and a fourth side, and a bottom surface, the distance from the at least one surface of the body to the bottom surface of the second opening determining a second depth, the second depth less than the thickness of the body, the distance from the first side to the third side determining a second width, the second width less than the width of the body, the distance from the second side to the fourth side determining a second length, the second length less than the length of the body but at least one quarter of the length of the body, the second volume determined by the second depth, second length and second width.

3. The stent of statement 2, the first therapeutic agent different than the second therapeutic agent.

4. The stent of statement 2, the first therapeutic agent having a first volume, the second therapeutic agent having a second volume, the first volume different than the second volume.

5. The stent of statement 2, the first therapeutic agent further deposited on the at least one surface of the body defining the first channel.

6. The stent of statement 5, the second therapeutic agent further deposited on the at least a portion of the at least one surface of the body defining the second channel.

7. The stent of statement 2, the at least one surface of the body comprising a first surface and a second surface, the first surface defining the first channel and the second surface defining the second channel.

8. The stent of statement 7, the first surface opposite of the second surface.

9. The stent of statement 8, the at least one surface further comprising a third surface, the third surface at an angle to the first and second surfaces, the first therapeutic agent further deposited on at least a first portion of the first surface of the body defining the first channel, the first portion of the first surface extending from the opening of the first channel to the third surface, the first therapeutic agent further deposited on at least a portion of the third surface, the second therapeutic agent further deposited on at least a first portion of the second surface of the body defining the second channel, the first portion of the second surface extending from the opening of the second channel to the third surface, the second therapeutic agent further deposited on at least a portion of the third surface.

10. The stent of statement 9, the first therapeutic agent the same as the second therapeutic agent.

11. The stent of statement 2, at least a portion of one of the first side, the second side, the third side, the fourth side and the bottom surface of the first channel being curvilinear.

12. The stent of statement 11, at least a portion of one of the first side, the second side, the third side, the fourth side and the bottom surface of the second channel being curvilinear.

13. The stent of statement 2, the first depth of the first channel different from the second depth of the second channel.

14. The stent of statement 2, at least a portion of the bottom surface being curvilinear.

15. The stent of statement 2, the first channel further having a second depth, the first depth being the distance from the first opening to a first portion of the bottom surface, the second depth being the distance from the first opening to a second portion of the bottom surface, the first depth greater than the second depth.

16. The stent of statement 15, the second channel further having a second depth, the first depth being the distance from the first opening to at least one first portion of the bottom surface, the second depth being the distance from the first opening to at least one second portion of the bottom surface, the first depth greater than the second depth.

17. The stent of statement 2, the first width of the first channel different from the second width of the second channel.

18. The stent of statement 2, the first length of the first channel different from the second length of the second channel.

19. The stent of statement 2, the first length at least half the length of the body.

20. The stent of statement 2, the first channel having a first surface area, the first surface area determined by the first side, second side, third side, fourth side, and bottom surface of the first channel, the second channel having a second surface area, the second surface area determined by the first side, second side, third side, fourth side, and bottom surface of the second channel, the first surface area different from the second surface area.

21. A stent, the stent comprising a plurality of members, each of the plurality of members having a body, the body having a first surface and a second surface, the body defining a plurality of holes extending from the first surface to the second surface, the stent further comprising a first therapeutic agent, the therapeutic agent deposited within the plurality of holes.

22. The stent of statement 21, wherein the plurality of holes extend at an oblique angle from the first surface to the second surface.

23. The stent of statement 21, the first therapeutic agent further deposited on the first surface.

24. The stent of statement 22, the stent further comprising a second therapeutic agent, the second therapeutic agent deposited on the first surface.

25. A stent, the stent comprising a first member, the first member having a first side, a second side and a third side, the first member having at least one delivery apparatus engaged thereto, the at least one delivery apparatus having a first portion, a second portion and a third portion, the first portion engaged to the first side of the first member, the second portion engaged to the second side of the first member, the third portion engaged to the third side of the first member, the second portion of the at least one delivery apparatus having at least one therapeutic agent engaged thereto.

26. A stent, the stent comprising a plurality of members, each of the plurality of members having a body, at least one of the bodies comprising at least one surface, a first region, a second region and at least one channel, the first region being porous and containing at least one therapeutic agent, the second region adjacent to at least a portion of the first region, the at least one channel extending from the first region to the at least one surface of the body.

27. The stent of statement 26, the at least one channel comprising a first channel and a second channel, the first channel having a different diameter than the second channel.

28. The stent of statement 26, the members manufactured of wire.

29. The stent of statement 26, the second region surrounding the first region.

30. The stent of statement 29, further comprising a third region, the first region surrounding the third region.

31. The stent of statement 30, the third region manufactured of a non-porous material.

32. The stent of statement 26, the second region manufactured of a polymer.

33. The stent of statement 26, further comprising a third region and a fourth region, first region between the second and third regions, the fourth region surrounding at least a portion of the first, second and third regions.

34. The stent of statement 33, the second and third regions made from a first material.

35. The stent of statement 34, the fourth region made from a second material, the second material different from the first material.

The following numbered statements characterize methods described above:

1. A method for engaging a therapeutic agent to at least one portion of a medical device having an uneven surface, comprising the steps of:
applying a polymer film to the at least one portion of the medical device, the polymer film having a softening temperature;
placing the medical device in a vacuum oven;
heating the medical device in the vacuum oven to a temperature just above the softening temperature of the polymer film; and
cyclically raising and lowering the pressure in the vacuum oven.

2. The method of statement 1, the medical device being a stent.

3. The method of statement 1, the polymer film being a therapeutic agent.

4. A method of engaging a therapeutic agent to a medical device, comprising the steps of:
providing a medical device, the medical device having an uneven surface, portions of the uneven surface lower than other portions of the uneven surface;
providing a therapeutic agent;
applying the therapeutic agent to the medical device above the melt flow temperature which is above the Tg for an amorphous polymer and above Tm for a semicrystalline polymer; and
allowing the medical device to cool such that the therapeutic agent can no longer flow.

5. The method of statement 4, wherein the medical device is a stent.

6. A method of engaging a therapeutic agent to a medical device, comprising the steps of:
providing a medical device, the medical device having a thickness and comprising a first channel, the first channel having an opening, a bottom surface, a first side, and a second side, the first channel having a first depth, the first depth the distance from the opening to the bottom surface of the first channel, the first depth less than the thickness of the medical device;
providing a therapeutic agent;
heating the medical device so that the first channel expands;
applying the therapeutic agent to the medical device; and
allowing the medical device to cool so that the first channel contracts.

7. The method of statement 6, the medical device being a stent.

8. A method of engaging a therapeutic agent to a medical device, comprising the steps of:
providing a medical device, the medical device having a thickness and comprising a first channel, the first channel having an opening, a bottom surface, a first side, and a second side, the first channel having a first depth, the first depth the distance from the opening to the bottom surface of the first channel, the first depth less than the thickness of the medical device;
providing a therapeutic agent;
depositing the therapeutic agent into the first channel; and
pressing the first side and the second side of the channel inwards so that the first and second sides of the channel partially cover the therapeutic agent deposited into the first channel.

9. The method of statement 8, the medical device being a stent.

10. A method of engaging a therapeutic agent to a medical device, comprising the steps of:
providing a medical device, the medical device having at least one indentation;
applying a layer of chromium with acid to the medical device;
depositing a material onto the surface of the medical device, wherein the material is selected from at least one member of the group consisting of a metal, a metal oxide, and any combination thereof;
depositing a polymer onto the surface of the medical device.

11. The method of statement 10, the medical device being a stent.

12. The method of statement 10, wherein the indentation is a channel or a hole.

13. The method of statement 10, wherein the metal is selected from at least one member of the group consisting of aluminum, iridium, titanium, tantalum, tungsten, niobium, gold, and platinum, and any combination thereof.

14. The method of statement 10, wherein the metal oxide is selected from at least one member of the group consisting of aluminum oxide, magnesium oxide, iron oxide, iridium oxide (Irox), iridium-iridium oxide (Ir—Irox), titanium oxide, titanium-iridium-iridium oxide (Ti—Ir—Irox), titanium-nitrogen oxide (TiNOx), titanium-titanium nitrogen oxide (Ti—TiNOx), tantalum oxide, tungsten oxide, niobium oxide, and any combination thereof.

15. The method of statement 10, wherein the material is deposited onto the surface of the stent by chemical vapor deposition, physical vapor deposition or pulsed laser deposition.

16. The method of statement 10, wherein the polymer is a therapeutic agent.

17. A method for manufacturing a stent with wells, comprising the steps of:
providing a stent tube;
providing a photomask, the mask having a design;
coating the stent tube with photoresist film;
holding the photomask over the stent tube and providing UV light so that the design on the photomask is transferred to the photoresist film coating the stent tube;
chemically processing the stent tube to remove portions of photoresist film not exposed to the UV light, thereby exposing areas of the stent tube;
cutting the stent tube into a stent;
electropolishing the stent thereby producing wells in the areas of the stent exposed; and
removing the portions of photoresist film exposed to the UV light.

18. A method for manufacturing a textured surface on at least a portion of a stent, comprising the steps of:
providing a stent, the stent having a surface;
providing a mask;
placing the mask over the surface of the stent;
etching the stent with a chemical; and
removing the mask.

19. A method for manufacturing a textured surface on at least a portion of a stent, comprising the steps of:
providing a stent, the stent having a surface;
providing a first mask;
placing the first mask on the surface of the stent;
depositing a material onto the surface of the stent, wherein the material is selected from at least one member of the group consisting of a metal, a metal oxide, and any combination thereof; and
removing the first mask.

20. The method of statement 19, wherein the metal is selected from at least one member of the group consisting of aluminum, iridium, titanium, tantalum, tungsten, niobium, gold, platinum, and any combination thereof.

21. The method of statement 19, wherein the metal oxide is selected from at least one member of the group consisting of aluminum oxide, magnesium oxide, iron oxide, iridium oxide (Irox), iridium-iridium oxide (Ir—Irox), titanium oxide, titanium-iridium-iridium oxide (Ti—Ir—Irox), titanium-nitrogen oxide (TiNOx), titanium-titanium nitrogen oxide (Ti—TiNOx), tantalum oxide, tungsten oxide, niobium oxide, and any combination thereof.

22. The method of statement 19, wherein the material is deposited onto the surface of the stent by chemical vapor deposition, physical vapor deposition or pulsed laser deposition.

23. The method of statement 19, further comprising the steps of:
providing a second mask;
placing the second mask over the surface of the stent;
etching the stent with a chemical; and
removing the second mask.

24. The method of statement 19, further comprising the steps of:
providing an energy source; and
using the energy source to remove at least a portion of the surface of the stent.

25. The method of statement 24, wherein the energy source is selected from at least one member of the group consisting of a laser, an ion beam, and any combination thereof.

The inventive stents 10, clamps 54/staples 56, and pins 58 may be made from any suitable biocompatible materials including one or more polymers, one or more metals or combinations of polymer(s) and metal(s). Examples of suitable materials include biodegradable or bioabsorbable materials that are also biocompatible. By biodegradable is meant that a material will undergo breakdown or decomposition into harmless compounds as part of a normal biological process. Suitable biodegradable materials include polylactic acid, polyglycolic acid (PGA), collagen or other connective proteins or natural materials, polycaprolactone, hylauric acid, adhesive proteins, co-polymers of these materials as well as composites and combinations thereof and combinations of other biodegradable polymers. Other polymers that may be used include polyester and polycarbonate copolymers. Examples of suitable metals include, but are not limited to, stainless steel, titanium, tantalum, platinum, tungsten, gold and alloys of any of the above-mentioned metals. Examples of suitable alloys include platinum-iridium alloys, cobalt-chromium alloys including Elgiloy and Phynox, MP35N alloy and nickel-titanium alloys, for example, Nitinol.

The inventive stents may be made of shape memory materials such as superelastic Nitinol or spring steel, or may be made of materials which are plastically deformable. In the case of shape memory materials, the stent may be provided with a memorized shape and then deformed to a reduced diameter shape. The stent may restore itself to its memorized shape upon being heated to a transition temperature and having any restraints removed therefrom.

The inventive stents may be created by methods including cutting or etching a design from a tubular stock, from a flat sheet which is cut or etched and which is subsequently rolled or from one or more interwoven wires or braids. Any other suitable technique which is known in the art or which is subsequently developed may also be used to manufacture the inventive stents disclosed herein.

In some embodiments the stent, the delivery system or other portion of the assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate. A more extensive list of therapeutic agents can be found in commonly assigned U.S. Patent Application Publication 2006/0045901, entitled Stents with Drug Eluting Coatings, hereby incorporated in its entirety.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the invention. Those skilled in the art may recognize other equivalents to the specific embodiment described herein which equivalents are intended to be encompassed by the claims attached hereto.

1. A stent comprising a first member and further comprising at least one mechanism to reduce delamination of a substance disposed onto at least a portion of a surface of the stent, the at least one mechanism being either:
a first mechanism, the first mechanism comprising a body, the body comprising a width, a first region and a second region, a first arm and a second arm, the first arm engaged to the body at the first region, the second arm engaged to the body at the second region, the first and second arms extending at oblique angles to the body, the first mechanism engaged to the first member; or
a second mechanism, the second mechanism comprising a first transverse portion and an elongated portion, at least a portion of the first transverse portion being engaged to the first member, the first transverse portion being at an oblique angle to the first member, the elongated portion being parallel to the first member.

2. The stent of claim 1, comprising a plurality of mechanisms to reduced delamination of a substance selected from the group consisting of:
the first mechanism;
the second mechanism; and
combinations thereof.

3. The stent of claim 1, the first member having a first surface, a second surface, and third and fourth surfaces extending between the first and second surfaces, wherein the first arm of the first mechanism is engaged to the first surface and the second arm of the first mechanism is engaged to the second surface.

4. The stent of claim 1, further comprising a substance, the substance being disposed on a surface of the first member and on a portion of the body of the first mechanism.

5. The stent of claim 1, the elongated portion of the second mechanism being disposed a first distance away from the first member.

6. The stent of claim 1, further comprising a substance, the substance being disposed on a surface of the first member and underneath the elongated portion of the second mechanism.

7. The stent of claim 1, further comprising a substance, the substance being disposed on at least a portion of the second mechanism.

8. The stent of claim 7, the second mechanism further comprising a first cap, the first cap being engaged to the first transverse portion and extending over a portion of the substance disposed on the second mechanism.

9. The stern of claim 8, wherein the first cap is parallel to the elongated portion.

10. The stent of claim 1, the elongated portion being a hollow tube.

11. The stent of claim 10, wherein a therapeutic agent is disposed within the hollow tube.

12. The stent of claim 11, the hollow tube having a first end and a second end, at least one of the ends being closed.

13. The stent of claim 12, the hollow tube further comprising at least one hole.

14. The stent of claim 1, wherein an end of the transverse portion is engaged to a surface of the first member.

15. The stent of claim 1, wherein a first portion of the transverse portion is disposed within a passageway in the first member, the first portion of the transverse portion engaging the second mechanism to the first member.

16. The stent of claim 15, wherein the transverse portion is a rivet.

17. The stent of claim 15, wherein a second portion of the transverse portion is disposed within a passageway through the elongated portion, the passageway being between a first end and a second end of the elongated portion.
